(19) United States
(12) Patent Application Publication
(10) Pub. No.: US 2020/0317813 A1
(43) Pub. Date: Oct. 8, 2020

(54) TREATMENT AND DIAGNOSIS OF NON-PROLIFERATIVE DIABETIC RETINOPATHY

(71) Applicant: Johann Wolfgang Goethe-Universitat Frankfurt am Main, Frankfurt am Main (DE)

(72) Inventors: Ingrid FLEMING, Frankfurt (DE); Timo FRÖMEL, Frankfurt (DE); Jiong HU, Frankfurt (DE); Rüdiger POPP, Dreieich (DE)

(21) Appl. No.: 16/303,940

(22) PCT Filed: May 24, 2017
(86) PCT No.: PCT/EP2017/062618

§ 371 Date: Nov. 21, 2018

(30) Foreign Application Priority Data
May 25, 2016 (EP) 102016109709.8

(51) Int. Cl. C07K 16/40; A61P 27/02; C12Q 1/34

(52) U.S. Cl. CPC C07K 16/40; C07K 2317/76; C12Q 1/34; A61P 27/02

(57) ABSTRACT

The present invention pertains to antagonists and inhibitors of soluble epoxide hydrolase (sEH) and 19,20-dihydroxydocosapentaenoic acid (19,20-DHDP) for use as a therapeutic in the treatment of eye disorders that are characterized by pericyte loss and motility. The invention is useful for treating the non-proliferative form of diabetic retinopathy. Furthermore provided are methods for monitoring or diagnosing diabetic retinopathy, in particular in subjects at risk of developing the disorder, for example diabetic patients.

### FIELD OF THE INVENTION

The present invention pertains to antagonists and inhibitors of soluble epoxide hydrolase (sEH) and 19,20-dihydroxydocosapentaenoic acid (19,20-DHDP) for use as a therapeutic in the treatment of eye disorders that are characterized by pericyte loss and motility. The invention is useful for treating the non-proliferative form of diabetic retinopathy. Furthermore provided are methods for monitoring or diagnosing diabetic retinopathy, in particular in subjects at risk of developing the disorder, for example diabetic patients.

### DESCRIPTION

Chronically high blood sugar from diabetes is associated with damage to the tiny blood vessels in the retina, leading to diabetic retinopathy. Diabetic retinopathy can cause blood vessels in the retina to leak fluid or hemorrhage (bleed), distorting vision. In its most advanced stage, new abnormal blood vessels proliferate (increase in number) on the surface of the retina, which can lead to scarring and cell loss in the retina.

Diabetic retinopathy is a severe complication of diabetes, affecting the vision of more than half of adult diabetics, and is the leading cause of blindness in adults in the United States. The mechanisms of diabetic retinopathy and therapeutic for treating it are the subject of extensive efforts. In a clinical setting, for example, laser photocoagulation and anti-angiogenic therapy represent state-of-the-art therapeutic strategies for inducing angiogenic regression and reduction of macular edema. Nevertheless, therapeutic challenges remain because many patients are unresponsive to current therapeutic approaches and/or because the state-of-the-art anti-angiogenic and photocoagulation therapies are accompanied by significant side-effects.

According to the National Eye Institute (NIH, USA), diabetic retinopathy progresses through four stages:

Mild non-proliferative retinopathy: Small areas of balloon-like swelling in the retina's tiny blood vessels, called microaneurysms, occur at this earliest stage of the disease. These microaneurysms may leak fluid into the retina which causes it to swell or to form deposits. Importantly, pericyte dropout precedes other major morphological changes of vessels observed in the retina (Cogan et al., Arch Ophthalmol. 1961; 66:366-78.) strongly suggesting that loss of pericytes may be a causal pathogenic event in this disease.

Moderate non-proliferative retinopathy: As the disease progresses, blood vessels that nourish the retina may swell and distort. They may also lose their ability to transport blood. Both conditions cause characteristic changes to the appearance of the retina and may contribute to diabetic macular edema.

Severe non-proliferative retinopathy: Many more blood vessels are blocked, depriving blood supply to areas of the retina. These areas secrete growth factors that signal the retina to grow new blood vessels.

Proliferative diabetic retinopathy: At this advanced stage, growth factors secreted by the retina trigger the proliferation of new blood vessels, which grow along the inside surface of the retina and into the vitreous gel, the fluid that fills the eye. The new blood vessels are fragile, which makes them more likely to leak and bleed. Accompanying scar tissue can contract and cause retinal detachment which can lead to permanent vision loss.

“Soluble epoxide hydrolase” (“sEH”) is an epoxide hydrolase which in many cell types converts fatty acid epoxides (e.g. the epoxyeicosatrienoic acids or “EETs” generated from arachidonic acid or epoxydocosapentaenoic acids “EDPs” generated from docosahexenoic acid) to dihydroxy derivatives e.g. dihydroxyeicosatrienoic acids or “DHETs” and dihydroxydocosapentaenoic acid or “DHDP”). The cloning and sequence of the murine sEH is set forth in Grant et al., J. Biol. Chem. 268(23):17628-17633 (1993). The cloning, sequence, and accession numbers of the human sEH sequence are set forth in Beetham et al., Arch. Biochem. Biophys. 305(1):197-201 (1993). NCBI Entrez Nucleotide accession number L05779 sets forth the nucleic acid sequence encoding the protein, as well as the 5′ untranslated region and the 3′ untranslated region. The evolution and nomenclature of the gene is discussed in Beetham et al., DNA Cell Biol. 14(1):61-71 (1995). The sEH represents a single highly conserved gene product with over 90% homology between rodent and human (Arand et al., FEBS Lett., 338:251-256 (1994)) and is only very distantly related to microsomal epoxide hydrolase (mEH) and hydrates a wide range of epoxides not on cyclic systems. In contrast to the role played in the degradation of potential toxic epoxides by mEH, sEH is believed to play a role in the formation or degradation of endogenous chemical mediators. Unless otherwise specified, as used herein, the terms “soluble epoxide hydrolase” and “sEH” refer to mammalian sEH. In preferred embodiments of the invention the term will refer to human sEH insofar the disclosed therapeutic or diagnostic applications pertain to human subjects.

It was therefore an object of the present invention to develop new therapeutic strategies for the treatment of diabetic retinopathy, in particular in early stages of the disease. Treatment of the disease in the proliferative stage requires possibly toxic medication and invasive surgery, and is often accompanied by reduced vision. Hence, the invention further intends to provide a diagnostic option for the detection of diabetic retinopathy in the non-proliferative state so that early treatment of the disease according to the invention is possible.

The above problem is solved in a first aspect by an inhibitor or antagonist of 19,20-dihydroxydocosapentaenoic acid (19,20-DHDP) for use in the treatment or prevention of an eye disease associated with the blood-retinal barrier.

The invention further provides a method for treating of an eye disease associated with the blood-retinal barrier comprising the administration of a therapeutically effective amount of an inhibitor or antagonist of 19,20-DHDP to a subject in need of the treatment.

In context of the present invention it was surprisingly found that 19,20-DHDP is a key factor in the early development of diabetic retinopathy. The reduction of 19,20-DHDP in the retina of subjects at risk of developing diabetic retinopathy by inhibition of the soluble epoxide hydrolase (sEH)—the main enzyme producing 19,20-DHDP—was shown to alleviate the onset of the disease in vivo. Hence, the present invention provides 19,20-DHDP, and the factors controlling 19,20-DHDP concentration, as a new therapeutic target for the treatment of eye disorders associated with the blood-retinal barrier, such as non-proliferative diabetic retinopathy.

Since the invention provides therapeutics that target diabetic retinopathy in its early stages, the invention is very useful for a preventive approach. In risk patients, the invention may be applied in order to counter the early development of a diabetic retinopathy. Patients at risk of developing an eye disorder treatable or preventable according to the invention are described herein below in more detail.

### Compounds

In context of the present invention the expression “inhibitor or antagonist of 19,20-DHDP” shall be understood to refer to a compound having any one of the following characteristics: a compound inhibiting the biochemical synthesis of 19,20-DHDP, a compound increasing the biochemical degradation of 19,20-DHDP, or a compound binding to 19,20-DHDP and inhibiting its biological functions in a cell, such as 19,20-DHDP cell-membrane integration or 19,20-DHDP localization in the lipid raft fraction of a cell membrane. Preferred inhibitor or antagonist of 19,20-DHDP are analogs of 19,20-DHDP that compared to 19,20-DHDP are chemically modified.

In one preferred embodiment of the invention the inhibitor or antagonist of 19,20-DHDP is a compound that inhibits or antagonizes an epoxide hydrolase, preferably soluble epoxide hydrolase (sEH). The inhibitor or antagonist of sEH is preferably a compound modulating the enzymatic conversion of 19,20 epoxydocosapentaenoic acid (19,20-EDP) into 19,20-DHDP. In context of the present invention a “modulating” is preferably an alteration of the equilibrium of the conversion reaction in direction of the educt (19,20-EDP). Such a compound in a further preferred embodiment is a compound directly binding to the sEH enzyme. Many sEH inhibitors are known in the art. Some examples of such inhibitors will be described in the following, however, these descriptions shall not be interpreted as being limiting.

The expressions “inhibitor or antagonist of sEH” and “sEH inhibitors” are used interchangeably herein. sEH inhibitors are well known in the art and include but are not limited to those disclosed in McElroy et al, J. Med. Chem., 46:1066-1080 (2003); U.S. Pat. Nos. 6,831,082, and 6,693,130, US Patent Application Publications 2007/0225283, 2006/0270609, 2008/0076770, 2008/0032978, 2008/153889, 2008/0207621, 2008/0207622, 2008/0200444, 2008/0200467, 2008/0227780, 2009/0023731, 2009/0082395, 2009/0082350, 2009/0082456 and 2009/0082423, U.S. patent application Ser. No. 12/426,136, and International patent applications WO2008/105968, WO2007/043652, WO2007/043653, WO2007/106705, WO2007/067836, WO2007/098352, WO2008/022171, WO2006/121719, WO2007/044491, WO2006/121684, WO2009/020960 and PCT/US2008/088244. All of the above listed publications, patents, patent applications are incorporated by reference in their entirety.

A variety of other chemical structures is known to have sEH inhibitory action. Such derivatives in which the urea, carbamate or amide is the pharmacophore (as used herein, “pharmacophore” refers to the section of the structure of a ligand that binds to the sEH), preferably in which the urea, carbamate or amide is covalently bound to both an adamantane and to a 12 carbon chain dodecane are particularly useful as sEH inhibitors in context of the herein disclosed invention. Derivatives that are metabolically stable are preferred, as they are expected to have greater activity in vivo. Selective and competitive inhibition of sEH in vitro by a variety of urea, carbamate, and amide derivatives is taught, for example, by Morisseau et al., Proc. Natl. Acad. Sci. U.S. A, 96:8849-8854 (1999), which provides substantial guidance on designing urea derivatives that inhibit the enzyme.

Derivatives of urea are transition state mimetics that form a preferred group of sEH inhibitors. Within this group, N,N′-dodecyl-cyclohexyl urea (DCU), is preferred as an inhibitor, while N-cyclohexyl-N′-dodecylurea (CDU) is particularly preferred. Some compounds, such as dicyclohexylcarbodiimide (a lipophilic diimide), can decompose to an active urea inhibitor such as DCU. Any particular urea derivative or other compound can be easily tested for its ability to inhibit sEH by standard assays, such as those discussed herein. The production and testing of urea and carbamate derivatives as sEH inhibitors is set forth in detail in, for example, Morisseau et al., Proc Natl Acad Sci (USA) 96:8849-8854 (1999).

N-Adamantyl-N′-dodecyl urea (“ADU”) is both metabolically stable and has particularly high activity on sEH. (Both the 1- and the 2-adamantyl ureas have been tested and have about the same high activity as an inhibitor of sEH.) Thus, isomers of adamantyl dodecyl urea are preferred inhibitors. It is further expected that N,N′-dodecyl-cyclohexyl urea (DCU), and other inhibitors of sEH, and particularly dodecanoic acid ester derivatives of urea, are suitable for use in the methods of the invention. Preferred inhibitors include: 12-(3-Adamantan-1-yl-ureido)dodecanoic acid (AUDA), 12-(3-Adamantan-1-yl-ureido)dodecanoic acid butyl ester (AUDA-BE), Adamantan-1-yl-3-{5-[2-(2-ethoxyethoxy)ethoxy]pentyl}urea.

A number of other sEH inhibitors which can be used in the methods and compositions are set forth in the International patent applications PCT/US2012/025074, PCT/US2011/064474, PCT/US2011/022901, PCT/US2008/072199, PCT/US2007/006412, PCT/US2005/038282, PCT/US2005/08765, PCT/US2004/010298 and U.S. Published Patent Application Publication 2005/0026844, each of which is hereby incorporated herein by reference in its entirety for all purposes.

U.S. Pat. No. 5,955,496 (the '496 patent) also sets forth a number of sEH inhibitors which can be used in the methods. One category of these inhibitors comprises inhibitors that mimic the substrate for the enzyme. The lipid alkoxides (e.g., the 9-methoxide of stearic acid) are an exemplar of this group of inhibitors. In addition to the inhibitors discussed in the '496 patent, a dozen or more lipid alkoxides have been tested as sEH inhibitors, including the methyl, ethyl, and propyl alkoxides of oleic acid (also known as stearic acid alkoxides), linoleic acid, and arachidonic acid, and all have been found to act as inhibitors of sEH.

In another group of embodiments, the '496 patent sets forth sEH inhibitors that provide alternate substrates for the enzyme that are turned over slowly. Exemplars of this category of inhibitors are phenyl glycidols (e.g., S, S-4-nitrophenylglycidol), and chalcone oxides. The '496 patent notes that suitable chalcone oxides include 4-phenylchalcone oxide and 4-fluourochalcone oxide. The phenyl glycidols and chalcone oxides are believed to form stable acyl enzymes.

Additional inhibitors of sEH suitable for use in the methods are set forth in U.S. Pat. No. 6,150,415 (the '415 patent) and U.S. Pat. No. 6,531,506 (the '506 patent). Two preferred classes of sEH inhibitors are compounds of Formulas 1 and 2, as described in the '415 and '506 patents. Means for preparing such compounds and assaying desired compounds for the ability to inhibit epoxide hydrolases are also described. The '506 patent, in particular, teaches scores of inhibitors of Formula 1 and some twenty sEH inhibitors of Formula 2, which were shown to inhibit human sEH at concentrations as low as 0.1 μM. Any particular sEH inhibitor can readily be tested to determine whether it will work in the methods by standard assays. Esters and salts of the various compounds discussed above or in the cited patents, for example, can be readily tested by these assays for their use in the methods.

There are many pro-drugs possible, but replacement of one or both of the two active hydrogens in the ureas described here or the single active hydrogen present in carbamates is particularly attractive. Such derivatives have been extensively described by Fukuto and associates. These derivatives have been extensively described and are commonly used in agricultural and medicinal chemistry to alter the pharmacological properties of the compounds. (Black et al, Journal of Agricultural and Food Chemistry, 21(5):747-751 (1973); Fahmy et al, Journal of Agricultural and Food Chemistry, 26(3):550-556 (1978); Jojima et al, Journal of Agricultural and Food Chemistry, 31(3):613-620 (1983); and Fahmy et al, Journal of Agricultural and Food Chemistry, 29(3):567-572 (1981).)

Such active proinhibitor derivatives are within the scope of the present invention, and the just-cited references are incorporated herein by reference. Without being bound by theory, it is believed that suitable inhibitors mimic the enzyme transition state so that there is a stable interaction with the enzyme catalytic site. The inhibitors appear to form hydrogen bonds with the nucleophilic carboxylic acid and a polarizing tyrosine of the catalytic site.

In some embodiments, the inhibitor of sEH is selected from the group consisting of: 3-(4-chlorophenyl)-1-(3,4-dichlorphenyl)urea or 3,4,4′-trichlorocarbanilide (TCC; compound 295); 12-(3-adamantan-1-yl-ureido) dodecanoic acid (AUDA; compound 700); 1-adamantanyl-3-{5-[2-(2-ethoxyethoxy)ethoxy]pentyl]}urea (AEPU; compound 950);) 1-(1-acetypiperidin-4-yl)-3-adamantanylurea (APAU; compound 1153); trans-4-[4-(3-Adamantan-1-yl-ureido)-cyclohexyloxy]-benzoic acid (tAUCB; compound 1471); cis-4-[4-(3-Adamantan-1-yl-ureido)-cyclohexyloxy]-benzoic acid (cAUCB; compound 1686); 1-(1 methylsulfonyl-piperidin-4-yl)-3-(4-trifluoromethoxy-phenyl)-urea (TUPS; compound 1709); trans-4-{4-[3-(4-Trifluoromethoxy-phenyl)-ureido]-cyclohexyloxy}-benzoic acid (tTUCB; compound 1728); 1-trifluoromethoxyphenyl-3-(1-propionylpiperidin-4-yl) urea (TPPU; compound 1770); 1-(1-ethylsulfonyl-piperidin-4-yl)-3-(4-trifluoromethoxy-phenyl)urea (TUPSE; compound 2213); 1-(1-(cyclopropanecarbonyl)piperidin-4-yl)-3-(4-(trifluoromethoxy)phenyl)urea (CPTU; compound 2214); trans-N-methyl-4-[4-(3-Adamantan-1-yl-ureido)-cyclohexyloxy]-benzamide (tMAUCB; compound 2225); trans-N-methyl-4-[4-((3-trifluoromethyl-4-chlorophenyl)-ureido)-cyclohexyloxy]-benzamide (tMTCUCB; compound 2226); cis-N-methyl-4-{4-[3-(4-trifluoromethoxy-phenyl)-ureido]-cyclohexyloxy}-benzamide (cMTUCB; compound 2228); and 1-cycloheptyl-3-(3-(1,5-diphenyl-1H-pyrazol-3-yl)propyl)urea (HDP3U; compound 2247). In particular preferred are tAUCB and TPPU as inhibitors of sEH.

Further useful as sEH inhibitors for the applications of the present invention are dual modulators of sEH and Peroxisome proliferator-activated receptor (PPAR)-gamma. Such dual modulators are disclosed in U.S. provisional patent application No. 62/188,010, and Blöcher R, Lamers C, Wittmann S K, Merk D, Hartmann M, Weizel L, Diehl O, Brüggerhoff A, Boß M, Kaiser A, Schader T, Göbel T, Grundmann M, Angioni C, Heering J, Geisslinger G, Wurglics M, Kostenis E, Brune B, Steinhilber D, Schubert-Zsilavecz M, Kahnt A S, Proschak E. “N-Benzylbenzamides: A Novel Merged Scaffold for Orally Available Dual Soluble Epoxide Hydrolase/Peroxisome Proliferator-Activated Receptor γ Modulators”. J Med Chem. 2016 Jan. 14; 59(1):61-81. doi: 10.1021/acs.jmedchem.5b01239. Epub 2015 Dec. 25. PubMed PMID: 26595749; both references are incorporated herein in their entirety. The documents disclose a new chemical structure having a dual activity as sEH inhibitor and PPAR-gamma agonist. The compounds were designed by fusing the pharmacophores of a known sEH-inhibitor and a PPAR-gamma agonist. Such dual inhibitors are also known from La Buscato, E.; Blocher, R.; Lamers, C.; Klingler, F.-M.; Hahn, S.; Steinhilber, D.; Schubert-Zsilavecz, M.; Proschak, E. Design and Synthesis of Dual Modulators of Soluble Epoxide Hydrolase and Peroxisome Proliferator-Activated Receptors. Journal of medicinal chemistry 2012, 55 (23), 1077 1-1 0775, also incorporated by reference herein in its entirety.

Any of the aforementioned compounds useful in context of the present invention may be applied alone or as combinations. For example, in context of the present invention it may be preferred to combine a sEH-inhibitor with an 19,20-DHDP inhibitory analog.

Preferably, at concentrations of 100 μM, the inhibitor inhibits sEH activity by at least 50% while not inhibiting mEH activity by more than 10%. Preferred compounds have an IC₅₀ (inhibition potency or, by definition, the concentration of inhibitor which reduces enzyme activity by 50%) of less than about 100 μM. Inhibitors with IC₅₀ of less than 100 μM are preferred, with IC₅₀ of less than 75 μM being more preferred and, in order of increasing preference, an IC₅₀ of 50 μM, 40 μM, 30 μM, 25 μM, 20 μM, 15 μM, 10 μM, 5 μM, 3 μM, 2 μM, 1 μM, 100 ηM, 10 ηM, 1.0 nM, or even less, being still more preferred. Assays for determining sEH activity are known in the art. The IC₅₀ determination of the inhibitor can be made with respect to an sEH enzyme from the species subject to treatment.

### Target Disorders

The herein disclosed compounds are useful in context of the treatment of eye disorders that are associated with the blood-retinal barrier, meaning that the disorder is characterized by a pathological damage of the blood-retinal barrier. In particular retinal disorders characterized by increased pericyte motility and/or pericyte loss in a retina, which induces dissolution of the blood-retinal barrier, are treatable by the compounds and methods of the invention.

A target retinal disease of the present invention may be any retinal disease as long as it is a disease involving the degeneration, impairment or destruction of a pericyte of the retina, or a disease resulting from the degeneration, impairment or destruction of a pericyte of the retina. Examples of these diseases include retinitis pigmentosa, age-related macular degeneration, diabetic retinopathy, retinal detachment, diabetic maculopathy, hypertensive retinopathy, retinal vascular occlusion (retinal artery occlusion; retinal vein occlusion such as central retinal vein occlusion and branch retinal vein occlusion; etc.), retinal arteriosclerosis, retinal tear, retinal hole, macular hole, ophthalmorrhagia, posterior vitreous detachment, pigmented paravenous retinochoroidal atrophy, gyrate atrophy of the retina and choroid, choroideremia, crystalline retinopathy, retinitis punctata albescens, corneal dystrophy, cone dystrophy, central areolar choroidal dystrophy, Doyne's honeycomb retinal dystrophy, vitelliform macular dystrophy, cystoid macular edema, occult macular dystrophy, Stargardt disease, retinoschisis, central serous chorioretinopathy (central retinopathy), spinocerebellar ataxia type 7, familial exudative vitreoretinopathy, enhanced S-cone syndrome, angioid streaks, autosomal dominant optic atrophy, autosomal dominant drusen, familial drusen, acute zonal occult outer retinopathy, cancer-associated retinopathy, light damage, ischemic retinopathy, inflammation-induced retinal degenerative disease, etc.

Among these, a more suitable target disease is diabetic retinopathy, in particular an early stage diabetic retinopathy, such as a mild non-proliferative retinopathy, a moderate non-proliferative retinopathy and/or a severe non-proliferative retinopathy. In some preferred embodiments the target disease is not a proliferative diabetic retinopathy, and is not a glaucoma, nor in general a disease or pathology associated with a pathological increased pressure of the inner eye (vitreous).

The target disease of the present invention also includes a disease involving or resulting from the impairment of any of the constituent layers of the retina, i.e., the inner limiting membrane, the nerve fiber layer, the ganglion cell layer, the inner plexiform membrane, the inner nuclear layer, the outer plexiform layer, the outer nuclear layer, the external limiting membrane, the visual cell layer, and the retinal pigment epithelium layer. Particularly suitable target is a disease involving or resulting from the impairment of the blood-retinal barrier.

The target patient to whom the present invention is suitably applied is a patient with the above retinal disease. A patient or subject of the present invention is preferably a mammal, most preferably a human.

Such a patient in preferred embodiments has developed the target retinal disease as a secondary disorder, for example in context of a metabolic disorder. Therefore, the patient to whom the present invention is suitably applied is a patient suffering from, or at risk of developing, said eye disease associated with the blood-retinal barrier. Such a patient may for example suffer from a metabolic disorder, in particular from the metabolic syndrome and/or from diabetes types I or II. Furthermore, the patient is a patient that suffers from a chronic metabolic disorder. In a preferred embodiment the patient to be treated according to the herein described invention does not suffer from a pathological increased pressure of the inner eye, such as glaucoma.

In some embodiment a treatment in accordance to the present invention is performed concomitantly with a laser therapy, vitrectomy, and/or a concomitant or sequential treatment with a corticosteroid or an anti-angiogenic drug, preferably an anti-vascular endothelial growth factor (VEGF) drug, such as bevacizumab.

### Routes for Administration

The compounds of the present invention for their medical uses as described herein are preferably systemically or locally administered to a subject in need of such a treatment.

A systemic administration in context of the present invention refers to oral, rectal, and parenteral (i.e., intramuscular, intravenous, and subcutaneous) routes for administration. Generally, it will be found that when the compounds of the invention are administered orally a larger quantity of the active agent is required to produce the same effect as a smaller quantity given parenterally. In accordance with good clinical practice, it is preferred to administer the compounds at a concentration level that will produce effective therapeutic effect without causing any harmful or untoward side effects.

However, since many sEH inhibitors are known to cause significant side effects, a local administration at the retina is a preferred route for administration in context of this invention. The term “local administration” in context of the present disclosure shall comprise any administration locally at the eye. The administration to the eye of a patient includes injection into the vitreous or aqueous humor of the eye, or by intrabulbar injection, or by administration as eye drops or eye ointments. In some embodiments, the methods include the use of a local drug delivery device (e.g., a pump or a biocompatible matrix) to deliver the composition to the eye, such as coated contact lenses or similar devices. In particular preferred are all routes to target the compounds of the invention to the retina of a subject in need of a treatment according to the present invention.

The invention furthermore provides pharmaceutical compositions comprising the above compounds indicated as therapeutics for the retinal diseases described before. The pharmaceutical compositions of the invention comprise additionally a pharmaceutically acceptable excipient and/or carrier. The pharmaceutical compositions of the invention are for use in the treatment of an eye disorder as defined herein above.

Preferably, the compositions administered according to the present invention will be formulated as solutions, suspensions, emulsions and other dosage forms for topical administration. Aqueous solutions are generally preferred, based on ease of formulation, as well as a patient's ability to easily administer such compositions by means of instilling one to two drops of the solutions in the affected eyes. However, the compositions may also be suspensions, viscous or semi-viscous gels, or other types of solid or semi-solid compositions.

The compositions administered according to the present invention may also include various other ingredients, including but not limited to tonicity agents, buffers, surfactants, stabilizing polymer, preservatives, co-solvents and viscosity building agents. Preferred pharmaceutical compositions of the present invention include the inhibitor with a tonicity agent and a buffer. The pharmaceutical compositions of the present invention may further optionally include a surfactant and/or a palliative agent and/or a stabilizing polymer.

The “therapeutically effective dose” of the compounds of the invention in a composition for purposes herein is determined by such considerations as are known in the art. The dose must be effective to achieve improvement including but not limited to an improved course of disease, more rapid recovery, and improvement of symptoms, elimination of symptoms and other indicators as are selected as appropriate measures by those skilled in the art. The compounds of the invention can be administered in a single dose or in multiple doses.

In general, the active dose of compound for humans is in the range of from 1 ng/kg to about 20-100 mg/kg body weight per day, preferably about 0.01 mg/kg to about 2-10 mg/kg body weight per day, in a regimen of one dose per day or twice or three or more times per day for a single dose or multiple dose regimen.

### Diagnostics

Furthermore, the invention in a further aspect provides a method for diagnosing a non-proliferative diabetic retinopathy in a subject comprising determining the level of sEH, and/or 19,20-DHDP in a vitreous sample from the subject, wherein an elevated level of sEH and/or 19,20-DHDP in the sample from the subject compared to a control sample or value indicates a non-proliferative diabetic retinopathy.

There is furthermore provided a method for monitoring a subject for the development of a diabetic retinopathy, wherein the method comprises determining the level of sEH, and/or 19,20-DHDP in vitreous samples obtained from the subject at at least one earlier and at at least one later time point, wherein an increase of the level sEH, and/or 19,20-DHDP in the vit-reous sample obtained at the at least one later time point compared to the vitreous sample obtained at the at least one earlier time point indicates the development of a non-proliferative diabetic retinopathy in the subject.

In addition the invention provides diagnostic kits suitable for performing the diagnostic methods of the invention.

The diagnostic methods of the invention may in some embodiments be performed exclusively ex vivo or in vitro.

A subject to be diagnosed according to the invention is preferably a subject at risk of developing a diabetic retinopathy. A subject at risk of developing a diabetic retinopathy may be a subject suffering from a metabolic disease, such as diabetes type I or II.

The diagnostic approach of the present invention is of particular use for the detection of an early stage diabetic retinopathy. Thus, the diagnostic method is preferably applied to test a subject in which previously the presence of a late stage eye disorder, such as a proliferative diabetic retinopathy was already excluded.

The present invention will now be further described in the following examples with reference to the accompanying figures and sequences, nevertheless, without being limited thereto. For the purposes of the present invention, all references as cited herein are incorporated by reference in their entireties. In the Figures:

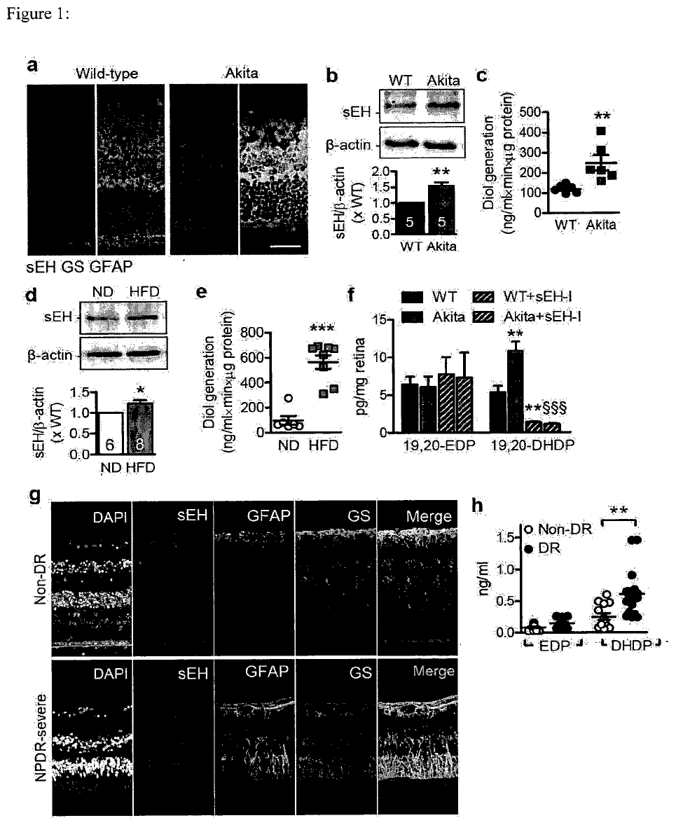

FIG. 1: Expression of sEH in retinas from diabetic mice and humans. (a) Retinal sEH (red) expression in 12 month old Ins2Akita (Akita) mice and their wildtype (WT) littermates and co-localization with glutamine synthetase (GS; green). Comparable results were obtained in retinas from 5 additional animals in each group; glial fibrillary acidic protein (GFAP; blue), bar=50 μm. (b) sEH expression in retinas from 12 month old Ins2Akita (Akita) mice and their wild-type (WT) littermates. (c) sEH activity determined by the generation of 14,15-DHET from 14,15-EET in retinas from 12 month old Ins2Akita (Akita) mice and their wild-type (WT) littermates. (d-e) sEH expression and activity in retinas from wild-type mice fed a normal diet (ND) or a high fat diet (HFD) for 20 weeks determined by Western blotting (c) and sEH activity assay (d). (f) Retinal levels of 19,20-EDP and 19,20-DHDP (n=6 per group) in retinas from 12 month old Ins2Akita mice and their wild-type littermates treated with vehicle or the sEH inhibitor. (g) Immunohistochemistry showing levels of sEH (red), glutamine synthetase (green) and GFAP (blue) in human retinal sections. Comparable results were obtained in retinas from 16 additional samples in each group. (h) 19,20-EDP and 19,20-DHDP levels in vitreous humor from individuals with non-diabetic retinopathy (n=14) or diabetic retinopathy (n=17); *P<0.05, **P<0.01, ***P<0.001.

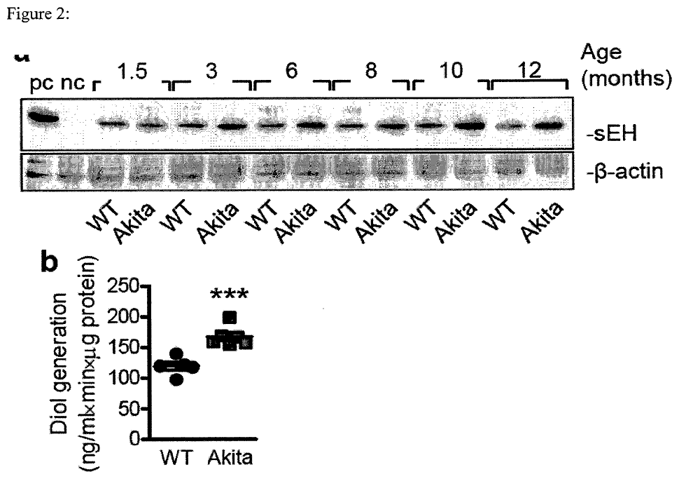

FIG. 2: Age-dependent increase in sEH expression and activity in retinas from Ins2Akita mice. (a) sEH expression in retinas from Ins2Akita (Akita) mice and their wild-type (WT) littermates. Comparable results were obtained in 3 additional experiments. Samples of liver from wild-type and sEH−/− mice were included as a positive control (pc) and negative control (nc) respectively. (b) sEH activity determined by the generation of 14,15-DHET from 14,15-EET in retinas from 6 month old Ins2Akita (Akita) mice and their wild-type (WT) littermates (n=5-6); *P<0.05, ***P<0.001.

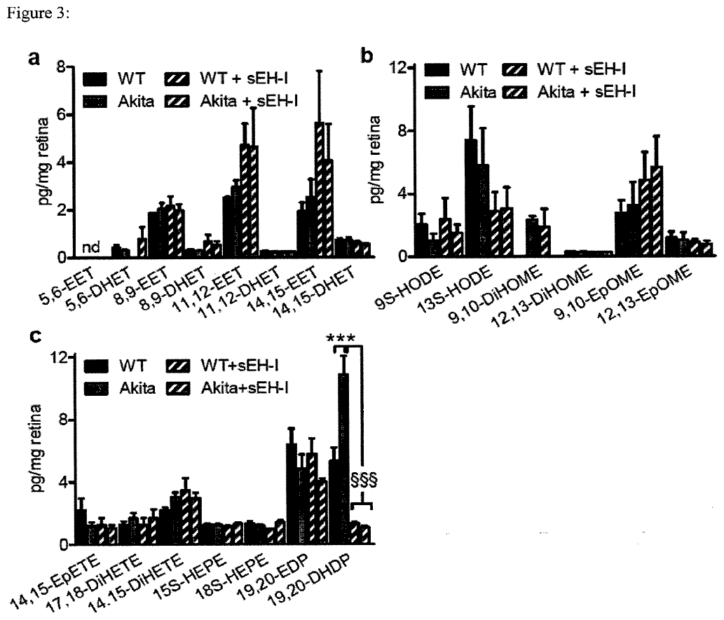

FIG. 3: Effect of diabetes and sEH inhibition on retina PUFA metabolite levels. Ins2Akita (Akita) mice and their non-diabetic wild-type (WT) littermates were treated with either vehicle (Veh; 0.3% ethanol) or sEH inhibitor (sEH-I) from 6 weeks to 12 months of age. (a) Arachidonic acid-derived products. (b) Linoleic acid-derived products. (c) Eicosapentaenoic acid- and docosahexaenoic acid-derived products. The data represent n=6 samples, with each sample representing a pool of retinas from 5 different animals; ***P<0.001 versus wild-type, § § § P<0.001 versus WT/sEH-I and Akita/sEH-I. EET, epoxyeicosatrienoic acid; DHET, dihydroxyeicosatrienoic acid; HODE, hydroxyoctadecadienoic acid; diHOME, dihydroxyoctadecenoic acid; EpOME, epoxyoctadecenoic acid; EpETE, epoxyeicosatetraenoic acid; diHETE, dihydroxyeicosatetraenoic acid; HEPE, hydroxyeicosapentaenoic acid; EDP, epoxydocosapentaenoic acid; DHDP, dihydroxydocosapentaenoic acid; nd=not detectable.

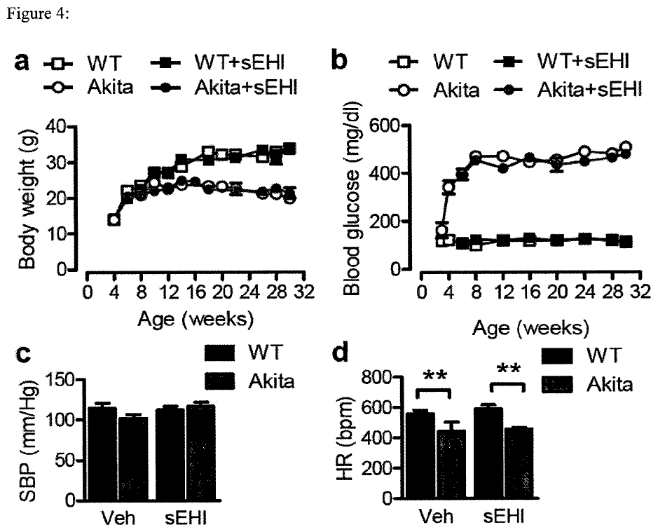

FIG. 4: Effect of chronic sEH inhibitor treatment on in vivo parameters. Wild-type (W) and Ins2Akita (Akita) littermates were treated with vehicle (Veh; 0.3% ethanol) or the sEH inhibitor (sEH-I) from the age of 6 weeks. (a) Body weight and (b) fasting blood glucose were recorded at monthly intervals. (c) Systolic blood pressure (SBP) and (d) heart rate were assessed at the age of 12 months. The data represent 8-10 animals per group; **P<0.01, ***P<0.001 versus wild-type.

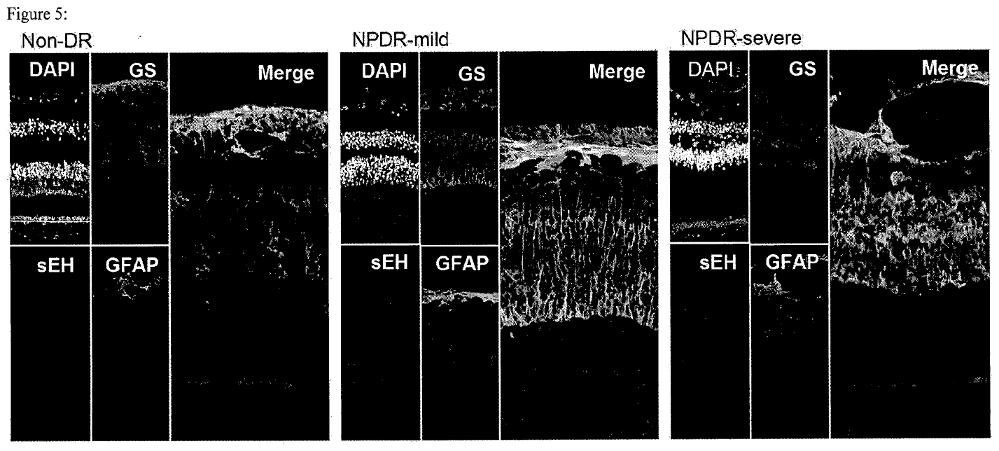

FIG. 5: sEH expression and activity in human retinas. sEH expression (red) in retinas from patients classed as having no diabetic retinopathy (non-DR), mild non proliferative diabetic retinopathy (NPDR) or severe NPDR. Glutamine synthetase (GS, green), glial fibrillary acidic protein (GFAP, blue) and DAPI (white). Comparable results were obtained in retinas from 5-6 additional samples in each group.

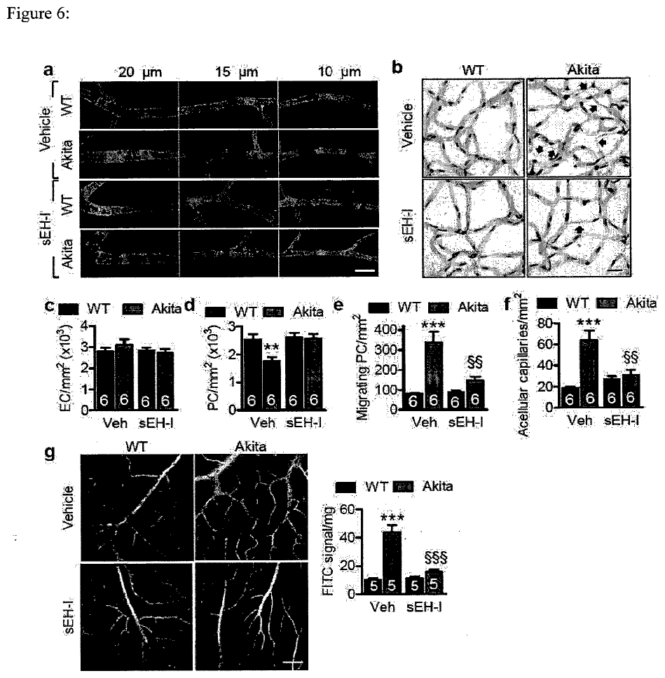

FIG. 6: Consequences of sEH inhibitor treatment on the development of diabetic retinopathy in mice. Ins2Akita (Akita) mice and their non-diabetic wild-type (WT) littermates were treated with either vehicle (Veh; 0.3% ethanol) or the sEH inhibitor trans-4-[4-(3-adamantan-1-ylureido)cyclohexyloxy]-benzoic acid (sEH-I) from 6 weeks to 12 months of age. (a) Smooth muscle actin staining showing pericyte coverage in vessels of 20 μm, 15 μm and 10 μm diameter; bar=50 mm. Comparable results were obtained in retinas from 5 additional animals in each group. (b) Representative images of periodic acid-schiff and hematoxylin staining of retinal digest preparations; bar=20 μm. Acellular capillaries are marked by arrows. (cf) Quantitative retinal morphometry images shown in b i.e., endothelial cells (c), pericytes (d), migrating pericytes (e), and acellular capillaries (f). (g) FITC-BSA fluorescence in retinas (bar=50 μm), and quantification of FITC-BSA after 2 hours washout with PBS; **P<0.01, ***P<0.001 versus wild-type/vehicle, §§ P<0.01, § § § P<0.001 versus Akita/Veh.

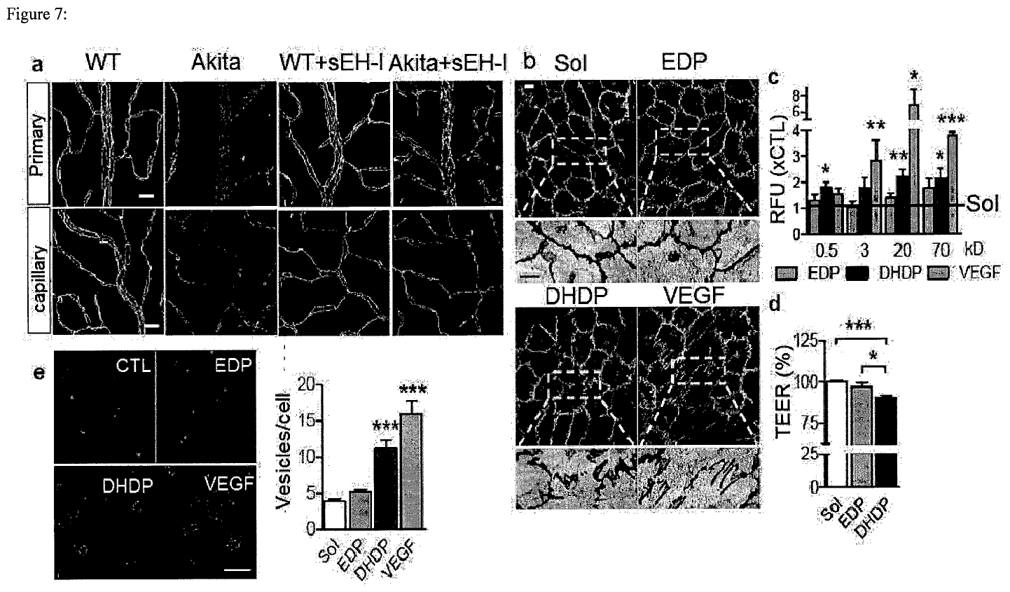

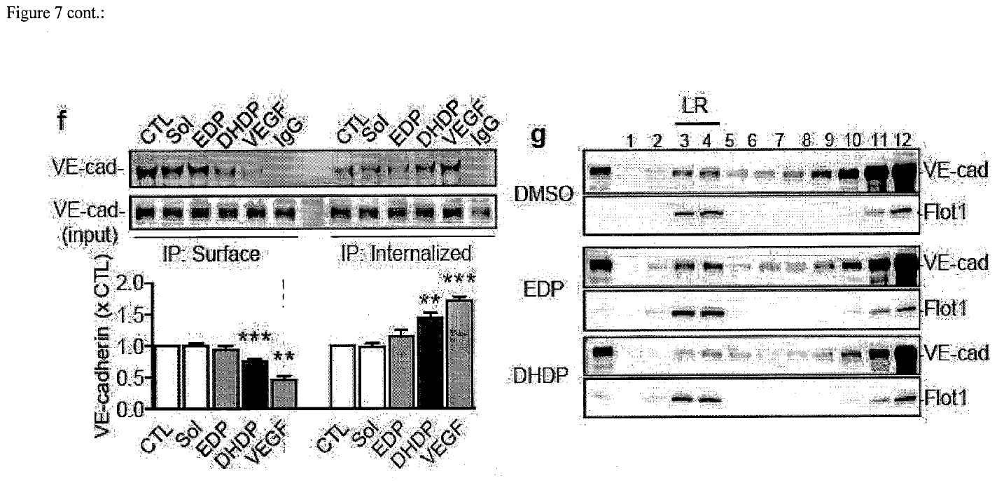

FIG. 7: Effect of 19,20-DHDP on endothelial cell permeability and the internalization of VE-cadherin. (a) VE-cadherin staining of the primary vascular layer and second capillary layer in retinas from 12 month old Ins2Akita (Akita) mice and wildtype (WT) littermates (bar=50 μm). Similar observations were made with 4 additional animals in each group. (b) VEcadherin staining of human endothelial cells treated with solvent (Sol; 0.03% DMSO), 19,20-EDP (3 μmol/L) or 19,20-DHDP (3 μmol/L) for 3 hours. The white rectangles indicate the areas magnified in the lower panels. (C) Permeability of human endothelial cells to dextran with different molecular masses after treatment with solvent, 19,20-EDP or 19,20-DHDP for 24 hours (n=4 independent cell preparations and 5 repetitions per experiment). (d) Transendothelial electrical resistance (TEER) in murine brain endothelial cells treated with solvent, 19,20-EDP (3 μmol/L), 19,20-DHDP (3 μmol/L) or VEGF (20 ng/ml) for 24 hours (n=4 independent cell batches, each studied in triplicate). (e) Internalized VE-cadherin in human endothelial cells treated with solvent, 19,20-EDP, 19,20-DHDP or VEGF for 3 hours and visualized following acid wash (n=4 different cell batches). Experiments were performed in the presence of the sEH inhibitor t-AUCB (10 μmol/L). (0 Surface and internalised VE-cadherin in endothelial cells treated with solvent, 19,20-EDP, 19,20-DHDP or VEGF for 3 hours in the presence of t-AUCB (n=4 different cell batches). (g) Distribution of VE-cadherin (VE-cad) and flotillin 1 (Flot1) in lipid rafts (LR; fractions 4 and 5) after treatment with solvent, 19,20-EDP or 19,20-DHDP. Comparable results were obtained in 3 additional experiments each using different cell batches. *P<0.05, **P<0.01, ***P<0.001 versus solvent.

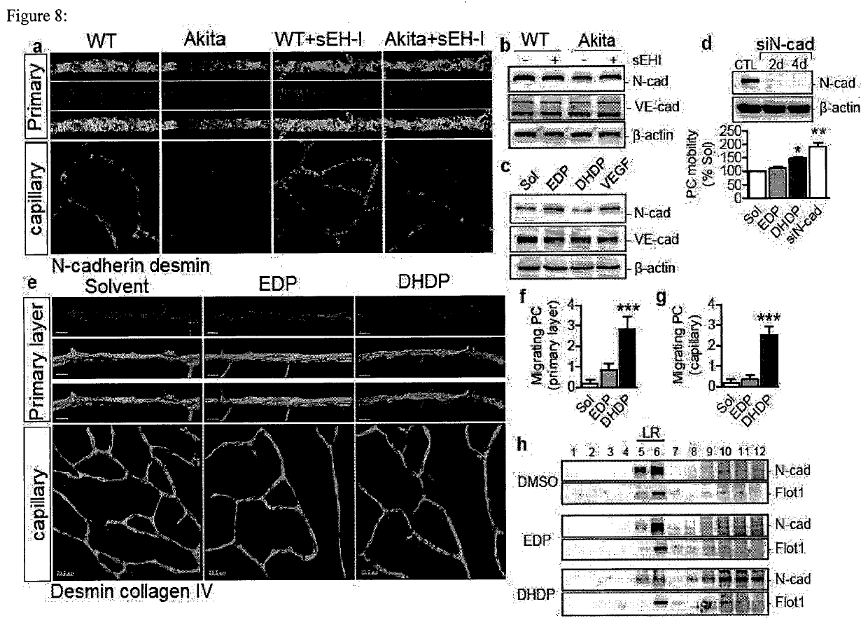

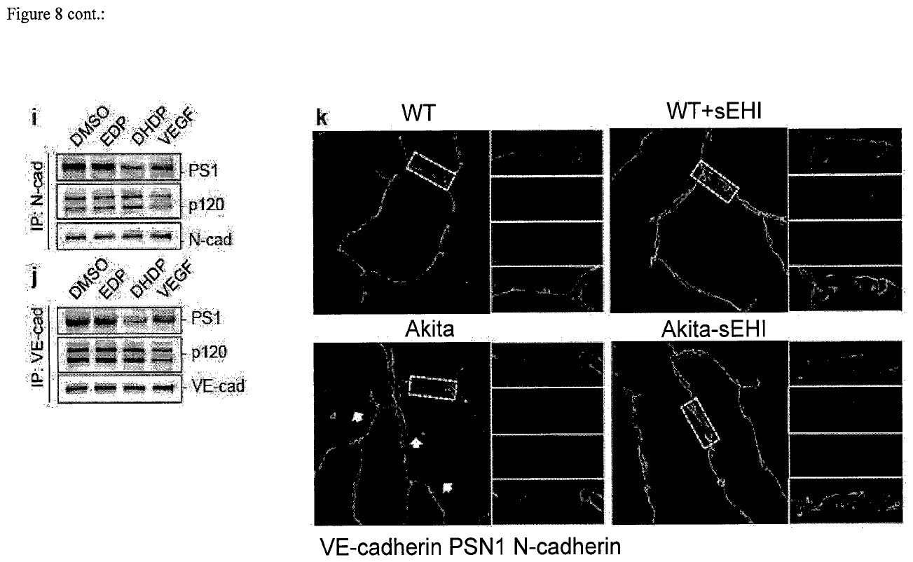

FIG. 8: Effect of 19,20-DHDP on N-cadherin internalization and pericyte drop-off (a) N-cadherin (green) and desmin (red) immunostaining in retinas from 12 month old diabetic Ins2Akita (Akita) mice and their non-diabetic wild-type (WT) littermates. Similar results were obtained in 5 additional animals in each group; bar=20 μm. (b) N-cadherin expression in retinas from Ins2Akita mice and wild-type littermates treated with or without sEH inhibitor. Similar results were obtained in 5 additional animals in each group. (c) VEcadherin and N-cadherin expression in endothelial cell-pericyte co-cultures after treatment with solvent (Sol, 0.03% DMSO), 19,20-EDP (3 μmol/L), 19,20-DHDP (3 μmol/L), or VEGF (20 ng/ml) for 24 hours. Similar results were obtained in 3 different cell batches. (d) Pericyte mobility on an endothelial cell monolayer after treatment with solvent, 19,20-EDP, 19,20-DHDP or siRNA directed against Ncadherin (siN-cad); n=3 per group. (e) Retinas from 7 month old wild-type mice were treated with solvent (0.05% DMSO), 19,20-EDP (5 μmol/L) or 19,20-DHDP (5 μmol/L) in an ex vivo organ culture system for 4 days. Images show the surface reconstitution of desmin (red) and collagen IV (green) signals from the primary vascular layer and the second capillary layer. (f-g) Quantification of migrating pericytes shown F (n=6 per group). (h) Distribution of N-cadherin (N-cad) and flotillin 1 (Flot1) in lipid rafts (LR; fractions 5 and 6) after treatment with solvent, 19,20-EDP or 19,20-DHDP. Similar results were obtained in 3 additional cell batches. (i-j) Association of PS1 and p120 with N-cadherin (i) and VE-cadherin (j) immunoprecipitated from endothelial cell-pericyte co-cultures after treatment with solvent, 19,20-EDP, 19,20-DHDP or VEGF for 24 hours. Similar results were obtained in 3 additional experiments. (k) VE-cadherin (green), PS1 (red) and Ncadherin (blue) immunostaining in retinas from 12 month old diabetic Ins2Akita (Akita) mice and their non-diabetic wild-type (WT) littermates treated with vehicle or sEH inhibitor. Arrows indicate disrupted VE-cadherin patterns along vasculature in Ins2Akita mice. Boxes highlight areas magnified on the right-hand panels. Similar results were obtained in 5 additional animals in each group; P<0.05, **P<0.01, ***P<0.001 versus solvent.

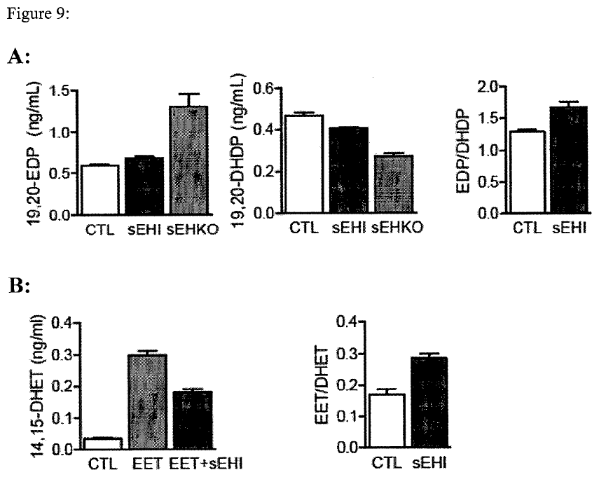

FIG. 9: Intravitreal injection of sEH inhibitor. (A) Ten week old C57 BL6 mice were treated with the sEH inhibitor TPPU (10 μmol/L administered intravitreally, total volume 1 μL), once per day for 3 days. Thereafter, retinas were isolated and processed for lipid profiling (n=3); (B) Ten week old C57 BL6 mice were treated with the sEH inhibitor TPPU (10 μmol/L administered intravitreally, total volume 1 μL), once per day for 3 days. To assay sEH activity, 14.15-EET (10 μmol/L total volume 1 μL, 1 hour) was injected following by retina isolation and lipid profiling.

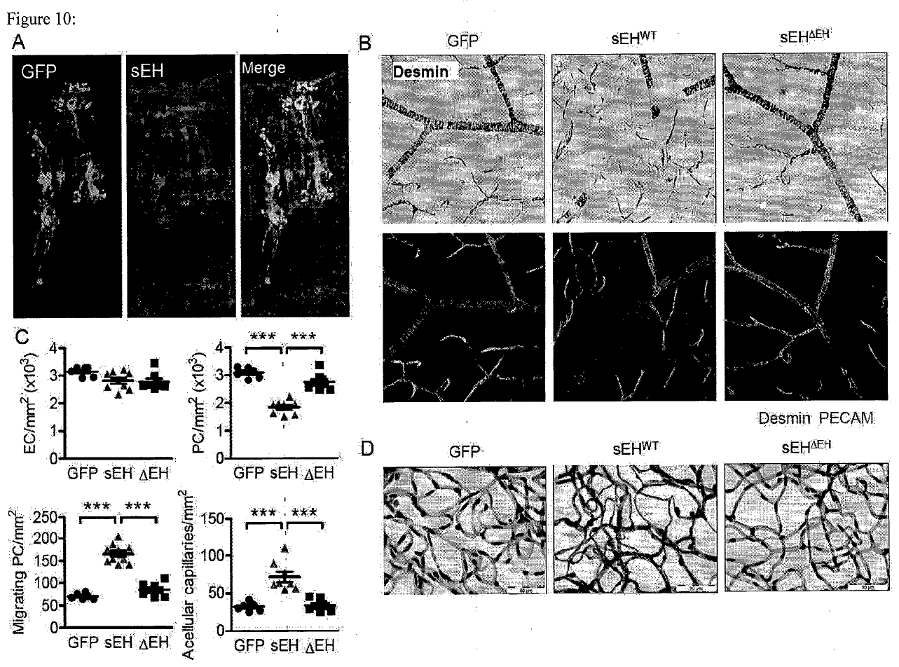

FIG. 10: Adenovirus mediated overexpression of sEH in murine retinas induces a retinopathy phenotype. (A) Representative images of sEH immunostaining in retinas 7 days after intravitreal injection of an adenovirus encoding the wild-type (WT) sEH together with GFP. Note the selective and robust expression of sEH in Müller cells. (B) Representative images of desmin and PECAM staining in murine retinas after intravitreal injections of adenoviruses encoding GFP, wild-type sEH (sEH^{WT}), or the epoxide hydrolase dead sEH mutant (sEH^{ΔEH}). (C) Quantitative analysis of endothelial cell (EC) and pericyte (PC) numbers and retinal vessel morphometry after intravitreal injection of adenovirus. (D) Representative images of retinal digest preparations from mice retinas that received intravitreal injections of adenoviruses encoding GFP, sEH^{WT} or sEH^{ΔEH}.

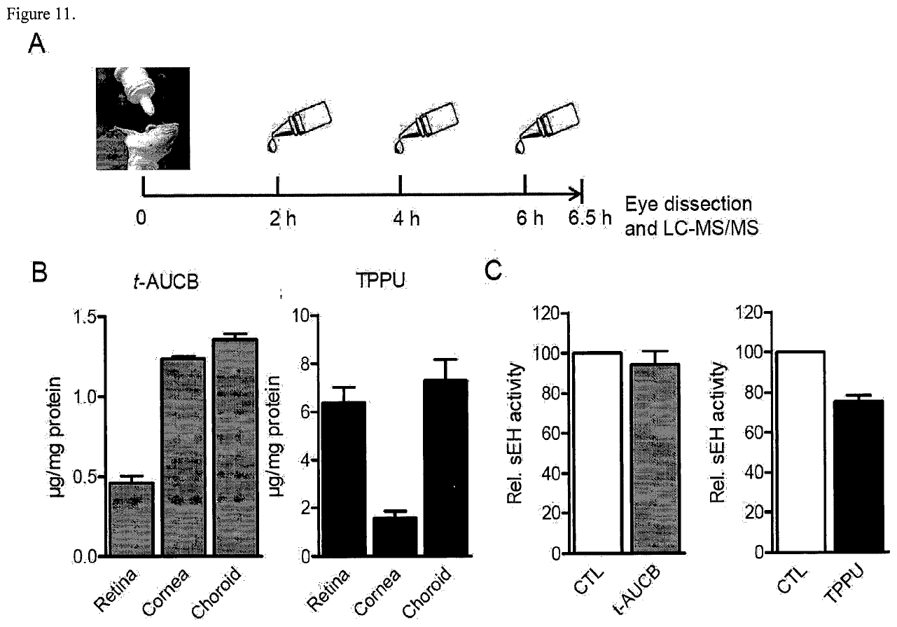

FIG. 11: Topical application of sEH inhibitors in vivo. (A) One drop (10 μl, of a 1 mg/ml solution) of sEH inhibitor (either t-AUCB or TPPU) was applied every 2 hours over the course of a 6 hour experiment. Thirty minutes after the last application, eyes were enucleated and dissected to obtain the cornea, retina, and eye cup (sclera and choroid). (B) The concentration of sEH inhibitor in the eye was determined by LC-MS/MS. (C) The relative sEH activity in the retina after topical application of sEH inhibitors.

### EXAMPLES

### Materials and Methods

### Materials

The sEH inhibitor trans-4-[4-(3-adamantan-1-ylureido)cyclohexyloxy]-benzoic acid (t-AUCB) was synthesised as described. Cell culture media were purchased from Gibco (Invitrogen, Karlsruhe, Germany), 19,20-epoxydocosapentaenoic acid and 19,20-dihydroxydocosapentaenoic acid were obtained from Cayman Europe (Tallinn, Estonia).

### Animals and In Vivo Treatment.

Ins2^{Akita} (C57BL/6-Ins2^{Akita}/J) mice carrying a mutation in the insulin 2 gene were obtained from The Jackson Laboratory (Bar Harbor, Me.). The colony was generated by breeding a C57BL/6J inbred female with a heterozygous male. In the present study exclusively male animals were studied—usually littermates. At the age of 6 weeks animals were treated with either vehicle (0.3% ethanol) or the sEH inhibitor (t-AUCB, 2 mg/L) in the drinking water for a further 10 months. All animals were housed in conditions that conform to the Guide for the Care and Use of Laboratory Animals published by the U.S. National Institutes of Health (NIH publication no. 85-23). Both the University Animal Care Committee and the Federal Authority for Animal Research at the Regierungspräsidium Darmstadt (Hessen, Germany) approved the study protocol (# F28/38). For the isolation of organs, mice were sacrificed using 4% isoflurane in air and subsequent exsanguination.

### Cell Culture and In Vitro Treatment

Murine brain endothelial cells were isolated and cultured as previously described^{29,30} and human brain vascular pericytes were purchased from ScienCell research laboratories (Berlin, Germany). Human umbilical vein endothelial cells were isolated and purified using VE-cadherin (CD144) antibody-coated magnetic beads (Dynal Biotech, Hamburg, Germany) and cultured as described³¹. The human umbilical cords were obtained from local hospitals in Frankfurt am Main, and the use of human material in this study conforms to the principles outlined in the Declaration of Helsinki. The isolation of human cells was approved by the ethics committee at the Goethe-University, Frankfurt, Germany.

### Human Samples

Retina Samples.

Eyes isolated during autopsy and embedded in paraffin were obtained from the Wilmer Eye Institute Ocular Pathology Archives with approval from the Johns Hopkins School of Medicine Institutional Review Board and the Eye Bank of the Center of Ophthalmology with approval from the Ethics Committee of the University of Cologne (reference number 14-247). and followed the tenets of the Declaration of Helsinki. Tissues were rehydrated and embedded in paraffin. Sections (5 μm) were cut on the horizontal level of the optic nerve and mounted on coated microscopic slides.

Vitreous Humor.

The vitreous samples for the lipid analysis were obtained from 17 patients undergoing vitrectomy for proliferative diabetic retinopathy and 14 patients with macula disease not related diabetes. All samples were collected by pars plana vitrectomy, were centrifuged at 13,000 rpm at 4° C. for 15 minutes and archived at −80° C. until further use. The study followed the tenets of the Declaration of Helsinki and was approved by Henan Eye Institute Clinical Research Ethics committee under the approval number HNEECKY-2015(3).

### Immunohistochemistry and Immunofluorescence

Retinal Whole Mount.

Retinas for whole-mount were fixed in 4% PFA for 2 hours at room temperature, or overnight at 4° C. After fixation, retinas were blocked and permeabilized in blocking buffer (1% BSA and 0.5% Triton X-100 in PBS) overnight at 4° C. For mouse antibodies, samples were washed and blocked with mouse IgG blocking reagent as instructed by the manufacturer (MKB-2213 Vector, CA, USA) for 1 hour at room temperature. Primary antibodies were diluted in blocking buffer and incubated overnight at 4° C. The following antibodies were used: VE-cadherin (1:200, AF1002 R&D systems, Abingdon, UK), α-smooth muscle actin-cy3 (1:500, C6198 sigma, Taufkirchen, Germany), N-cadherin (1:200, 33-3900 lifetechnologies, CA, USA), collagen IV (1:500, 1340-01 southern biotech, AL, USA), presenilin 1 (1:200, MAB1563 Millipore, Darmstadt, Germany) and desmin (1:500, ab15200 abcam, Cambridge, UK). For secondary detection Alexa Fluor-coupled secondary antibodies (1:200) were used. Cell nuclei were visualized with DAPI (0.2 ug/mL, D9542 Sigma). After antibody staining retinas were post-fixed with 4% PFA for 10 minutes before flat-mounting in mounting medium (Dako).

Retinal Cross Sections.

Immunofluorescence detection of sEH were performed in cryopreserved mouse tissue sections (10 μm) and paraffin-embedded human tissue (5 μm). The antibody against sEH (1:250) was kindly provided by Michael Arand (University of Zurich, Zurich, Switzerland). Antibodies against GFAP (1:1000), and GS (1:1000) were obtained from Millipore (Darmstadt, Germany).

Cultured Cells.

Cells were fixed with 4% PFA for 10 min at room temperature. After washing with PBS samples were blocked and permeabilized with 1% BSA, 5% horse serum, 0.5% Triton X-100 at room temperature for 2 hours or at 4° C. overnight before being exposed to primary antibody (4° C., overnight). After extensive washing and exposure to Alexa Fluor-coupled secondary antibody (1:200, 2 hours, room temperature) samples were mounted in fluorescent mounting medium and analyzed with a confocal microscope; Leica SP8 confocal microscope and LASX software (Wetzlar, Germany) or Carl Zeiss LSM-780 and ZEN software (Jena, Germany) as described⁶′³².

Retinal Digest Preparation

Retinal vascular preparations were performed using a pepsin-trypsin digestion method as previously described³³. Briefly, eyes were enucleated and immediately fixed in 4% PFA (PBS buffered, pH7.4) for two days and washed in distilled water for 1 hour at 37° C. A combined pepsin (5% pepsin in 0.2% hydrochloric acid for 1 hour)-trypsin (2.5% in 0.2 mol/L Tris/pH7.4 for 30 minutes) digestion was used to isolate the retinal vasculature. The samples were air-dried and stained with periodic acid Schiff and haematoxylin to highlight basement membranes and nuclei of capillary. The total number of endothelial cells, pericytes and migrating pericytes was counted in 10 randomly selected fields (×400 magnification) per retina using a Cell^{F} image system with a morphometric analyzing software. The total number of pericytes was counted in 10 randomly selected fields of the retina using an image analyzing system (XC10 Peltier-colled digital camera, Olympus Europa, Hamburg, Germany), and the numbers were normalized to the relative capillary density (number of cells per mm² capillary area). Subsequently, according to their localization within the capillary tree and their position relative to adjacent endothelial cells, pericytes with triangular nuclei in which and at least one lateral side of the triangular nuclei was longer than the basis in contact to the capillary migrating from capillaries into the extravascular interstitium, were defined as migrating pericytes. A cellular capillary segments were quantified using a integtration ocular with 100 grids (Olympus, 400× magnification) and the total numbers were normalized to mm² of retinal area. All samples were evaluated in a blinded fashion.

### sEH Activity Assay

Retinas were homogenized in RIPA lysis buffer (50 mmol/L Tris/HCL pH 7.5, 150 mmol/L NaCl, 10 mmol/L NaPPi, 20 mmol/L NaF, 1% sodium deoxycholate. 1% Triton X-100 and 0.1% SDS) and the homogenate was used to determine the sEH activity. Briefly, reactions were performed with 5 μg protein at 37° C. for 20 minutes in 100 μL of potassium phosphate buffer (100 mmol/L, pH 7.2). Reactions were started by the addition of 14,15-EET (10 μmon), stopped on ice and immediately extracted twice with ethyl acetate (0.7 mL) For LC/MS-MS analysis one tenth of the sample was spiked with a deuterated internal standard (14,15-EET-d8). After evaporation of the solvent in a vacuum block under a gentle stream of nitrogen the residues were reconstituted with 50 μL of methanol/water (1:1, v/v) and determined with a Sciex API4000 mass spectrometer operating in multiple reaction monitoring (MRM) mode as described^{34,35}. Chromatographic separation was performed on a Gemini C18 column (150×2 mm I.D., 5 μm particle size; Phenomenex, Aschaffenburg, Germany).

### Epoxide/Diol Profiling

Human vitreous (200 μL) or mouse retina lysates were mixed with 500 μL methanol and 300 μL 10 mol/L sodium hydroxide and deuterated internal standards. The samples were hydrolyzed for 30 minutes at 60° C. and then neutralized with acetic acid and adjusted to pH6.2. A solid phase extraction procedure using Agilent Bond-Elut-Certify II (Santa Clara, Calif., USA) was performed as described³⁶. The measurements were performed by LIPIDOMIX GmbH (Berlin, Germany) with a Triplequad LC-MS-MS instrument Agilent 6460/1200SL (Agilent Technologies, Waldbronn, Germany) equipped with a Phenomenex Kinetex Column (150 mm×2.1 mm, 2.6 μm, Phenomenex, Aschaffenburg, Germany). Chromatography was achieved under gradient conditions with acetonitrile/0.1% formic acid in water as the mobile phase, a flow rate of 0.3 mL/min and a run time of 16 minutes. The injection volume was 7.5 μL. After optimization the following MS-MS conditions were used: electrospray ionization (ESI) in negative mode, capillary voltage 3500 V, nozzle voltage 1500 V, drying gas 210° C./7 L/min, sheath gas 350° C./11 L/min and nebulizer pressure 30 psi.

### Pericyte Migration Assays

In Vitro Migration.

Human umbilical vein endothelial cells were isolated and cultured as described³⁷, seeded on μ-slides (ibidi, Martinsried, Germany) and grown to confluence. Pericytes were labeled with cell tracker green (Invitrogen) and added to the endothelial cell monolayer. Cells were treated with either solvent (0.03% DMSO), 19,20-EDP (3 μmol/L), 19,20-DHDP (3 μmol/L). Cells were incubated in an IncuCyte imaging system (Essen Bioscience) that took photographs automatically every 15 minutes for 48 hours. Pericyte movement on top of the endothelial cell monolayer were tracked manually and analyzed with Image J (NIH, Bethesda, Md., USA). In some experiments pericytes were treated with small interfering RNA directed against N-cadherin (sc-29403, Santa Cruz). Transfection was performed with lipofectamine 2000 (Invitrogen) following the manufacturer's instructions and a scrambled control siRNA was used as control.

Retinal Explants.

Eyes from 7 months old animals were enucleated and immersed in ice-cold HBSS containing penicillin (100 U/mL) and streptomycin (100 μg/mL). Retinas were carefully dissected under stereomicroscope and divided into four quadrants with four deep radial incisions. The explants were transferred onto tissue culture inserts (0.4 μm pore, Millipore; Cork, Ireland) with the retinal ganglion cell side facing up. The inserts were placed into the wells of a 6-well plate. A serum free retinal explant media (Neurobasal A, Invitrogen) supplemented with 2% B27 (Invitrogen), 1% N2 (Invitrogen), L-glutamine (0.8 mmol/L), penicillin (100 U/mL), and streptomycin (100 μg/mL) were added to the bottle of the wells and 3 μL of media was dropped on top of the retina to keep it moist. Retinal explant cultures were maintained in humidified incubators (37° C., 5% CO₂) and treated with either solvent (0.03% DMSO), 19,20-EDP (3 μmol/L) or 19,20-DHDP (3 μmon) in the presence of sEH inhibitor (t-AUCB, 10 mmol/L). After 4 days, retinas were fixed and processed as described above.

### Permeability Assays

Retinal Barrier Function.

Animals were anesthetized (Ketamine 100 mg/kg and Xylazine 10 mg/kg body weight) and injected (i.v.) with FITC-BSA (Sigma). After 2 hours, animals were killed and eyes were enucleated. After fixing with 4% PFA for 2 hours, retinas were dissected and flat mounted and analyzed with confocal microscopy. To quantify FITC-BSA leakage in the retina, some animals were perfused with pre-warmed PBS to remove FITC-BSA in the circulation followed by retina isolation. Thereafter, the FITC-BSA fluorescence signal in retinal homogenates was assessed (excitation λ485 nm, emission λ530 nm) using a plate reader (PerkinElmer, Hamburg, Germany).

Transendothelial Electrical Resistance (TEER).

Impedance measurements were performed using isolated murine brain microvascular endothelial cells with a cellZscope device (nano-Analytics) as described³⁸. After reaching confluence, indicated by a plateau in the TEER, cells were treated with either solvent (0.025% DMSO), 19,20-EDP (3 μmol/L) and 19,20-DHDP (3 μmol/L). Measurements were performed four times in quadruplicate with endothelial cells from four different cell preparations.

Dextran Permeability.

Permeability through the mouse brain endothelial cell monolayer was measured as described³⁹. Briefly, primary endothelial cells were plated (10⁵ cells/cm²) onto fibronectin-coated 24 well polyethylene terephthalate Transwell inserts (Greiner Bio-One, Frickenhausen, Germany) and cultured to confluency. Cells were treated with solvent, 19,20-EDP or 19,20-DHDP and after 24 hours dextrans of defined molecular mass and fluorescence i.e. 0.45 kDa Lucifer yellow-dextran (5 μmol/L, Sigma, excitation λ425 nm, emission λ 525 nm), 3 kDa dextran TXR (2.5 μmol/L, Invitrogen, excitation λ595 nm, emission λ625 nm), 20 kDa dextran TMR (5 μmol/L, Sigma, excitation λ550 nm, emission λ580 nm) and 70 kDa dextran FITC (2.5 μmol/L, Sigma, excitation λ490 nm, emission ζ520 nm) were added to the apical compartment. After 1 hour the transfer of dextrans to the lower compartment was assessed by a fluorescence reader (Tecan, Männedorf, Switzerland). The data are expressed as the percentage of permeability normalized to the permeability coefficient for the control conditions of untreated cells.

### VE- and N-Cadherin Internalization Assay

Internalization Assays.

VE-cadherin internalization was determined as described⁴⁰. Endothelial cells were cultured on culture slides (BD, Heidelberg, Germany) coated with crosslinked gelatin until confluent and starved with 2% FCS overnight. Cells were then incubated with antibody against extracellular domain of human VE-cadherin (clone BV6, ALX-803-305 Enzo Life Sciences, Lörrach, Germany) at 4° C. for 1 hour in MCDB131 with 1% BSA medium. Unbound antibody was removed by rinsing cells with ice-cold MCDB131 medium. After washing, cells were treated with solvent (0.03% DMSO), 19,20-EDP (3 μmol/L), 19,20-DHDP (3 μmol/L) or recombinant human vascular endothelial growth factor A (VEGF, 30 ng/ml) in the present of sEH inhibitor (t-AUCB, 10 mmol/L) at 37° C. for 3 hours. To block the degradation of internalized VE-cadherin chloroquine (300 μmol/L) was be added to the incubation medium. To assess internalized endogenous VE-cadherin cells were washed with ice-cold acid wash buffer (Hanks buffer, pH=2.7, containing 25 mmol/L glycine and 1% BSA) to assess total VE-cadherin cells were rinsed with ice-cold neutral washing buffer (Hanks buffer with 1% BSA, pH=7.4). Cells were fixed with 4% PFA for 15 minutes at 4° C. and then processed for immunofluorescence. To assess N-cadherin internalization, pericytes were incubated with an antibody against the extracellular domain of human N-cadherin (clone 8C11, 350802 BioLegend, CA. USA) at 4° C. for 1 hour in DMEM containing 1% BSA. After removing unbound antibodies with ice-cold DMEM medium, cells were treated with solvent (0.03% DMSO), 19,20-EDP (3 μmol/L) or 19,20-DHDP (3 μmol/L) in the present of sEH inhibitor (t-AUCB, 10 mmol/L) and leupeptin (100 μg/ml) at 37° C. for 3 hours. After rinsing with PBS, cells were fixed with 4% PFA for 15 minutes. Antibodies remaining on the cell surface were blocked with an excess of anti-mouse IgG Alexa 633-conjugated antibody (Life Technologies, 1:100) for 2 hours, before samples were rinsed with PBS and permeabilized with 0.5% Triton X-100. Endocytosed N-cadherin was visualized with anti-mouse IgG Alexa 546-conjugated antibody (Life Technologies, 1:400) and endosomes identified using early endosome antigen 1 (1:500, ab2900 abcam, Cambridge, UK). Samples were imaged with an SP8 confocal microscope (Leica) and internalized particles were quantified using ImageJ (NIH) software.

VE-Cadherin Immunoprecipitation.

Confluent cultures of human endothelial cells were incubated with antibodies recognizing the extracellular domains of human VE-cadherin (BV6 antibody) at 4° C. (1 hour) and then treated with 19,20-EDP or 19,20-DHDP (37° C., 3 hours). After acid wash to remove only the surface bound antibody, cells were homogenized with lysis buffer (20 mmol/L HEPES pH 7.5, 1.5 mmol/L MgCl₂, 5 mmol/L EGTA, 150 mmol/L NaCl, 1% Triton-X100, 0.5% glycerol). For surface VE-cadherin immunoprecipitation, endothelial cells were treated 19,20-EDP or 19,20-DHDP at 37° C. for 3 hours, and then incubated with VE-cadherin BV6 antibodies at 4° C. for 1 hour. After rinsing with ice-cold MCDB 131 medium to remove unbound antibody, cells were lysed with the same buffer as above. Following centrifugation at 17,000 g for 10 minutes, supernatants were incubated with protein G agarose (Pierce) for 2 hours. Samples were washed with lysis buffer and analyzed by SDS-PAGE.

### Endothelial Cell-Pericyte Co-Culture and Immunoprecipitation

Human umbilical vein endothelial cells were cultured on fibronectin coated dishes until confluent. Then pericytes were seeded on top of the endothelial cells (PC:EC=1:5) and cocultured for a further 3 days. After starving with 0.1% BSA overnight, co-cultures were treated with solvent (0.03% DMSO), 19,20-EDP (3 μmol/L), 19,20-DHDP (3 μmol/L) or VEGF (30 ng/ml) in the presence of the sEH inhibitor (t-AUCB, 10 mmol/L) at 37° C. for 10 hours. Cells were then lysed with HEPES buffer: 25 mmol/L HEPES pH 7.4, 150 mmol/L NaCl, complete protease inhibitor cocktail (Roche), and 1% digitonin (Sigma). Following centrifugation at 17,000 g for 10 minutes, supernatants (1 mg of protein) were pre-cleaned with protein A/G agarose (Pierce) for 30 mins. Supernatants were then incubated with anti-VE-cadherin (1:1000, AF1002 R&D systems, Abingdon, UK) or N-cadherin (1:1000, 33-3900 life technologies, CA, USA) antibodies at 4° C. for 2 hours and then treated with protein A/G agarose for a further 2 hours. Samples were washed with lysis buffer and analyzed by SDS-PAGE.

### Rt-Qpcr

Total RNA from retinas was extracted using an RNeasy kit (QIAGEN, Hilden, Germany), and equal amounts (1 μg) of total RNA was reverse transcribed (Superscript III; Invitrogen). Gene expression levels were detected using SYBR Green (Absolute QPCR SYBR Green Mix; Thermo Fisher Scientifc). The relative expression levels of the different genes studied was calculated using the 2^{-ΔΔCt} method with the 18S RNA as a reference.

### Small Interfering RNA

To knockdown N-cadherin in pericytes, small interfering (si) RNA directed against N-cadherin (sc-29403, Santa Cruz) was used. Transfection was performed with lipofectamine 2000 (Invitrogen) according to the manufacturer's instructions and a scrambled control siRNA was used as control.

### Immunoblotting

Retinas or cells were lysed in RIPA lysis buffer (50 mmol/L Tris/HCL pH 7.5, 150 mmol/L NaCl, 10 mmol/L NaPPi, 20 mmol/L NaF, 1% sodium deoxycholate. 1% Triton and 0.1% SDS) and detergent-soluble proteins were resuspended in SDS-PAGE sample buffer. Samples were separated by SDS-PAGE and subjected to Western blotting as described⁴¹. Membranes were blocked in 3% BSA in PBS, incubated with primary and horseradish peroxidase-conjugated secondary antibodies in blocking solution, and detection was performed with a Lumi-Light plus western blotting substrate (Roche).

### Lipid Rafts

Lipid rafts were isolated as described with modifications⁴². Briefly, cells were harvested by scraping and homogenized at 4° C. in sodium carbonate (0.5 mol/L, pH11) using a glass homogenizer, followed by sonication. Then, equal amounts of protein were adjusted to a final sucrose concentration of 45% (final volume, 4 mL) and transferred to 12 mL ultracentrifuge tubes. A discontinuous sucrose gradient was then formed by sequentially overlaying 4 mL of 35% and 4 ml, of 5% sucrose. Samples were subjected to ultracentrifugation (35,000 rpm, 4° C. for 20 hours) using a Beckman SW 41 rotor (Krefeld, Germany). After centrifugation, twelve 1 mL fractions were collected using a capillary tube connected to a peristaltic pump, and equal volumes of each faction were analyzed by SDS-PAGE using antibodies against flotillin 1 (1:2000, 610820 BD Biosciences, Heidelberg, Germany), VE-cadherin (1:1000, sc-28644 Santa Cruz, Darmstadt, Germany) and N-cadherin (1:1000, 33-3900 lifetechnologies, CA, USA).

### Statistical Analysis

Data are expressed as mean±SEM. Statistical evaluation was performed using Student's t test for unpaired data, one-way ANOVA followed by a Bonferroni t test or ANOVA for repeated measures where appropriate. Values of P<0.05 were considered statistically significant.

### Example 1: Soluble Epoxide Hydrolase (sEH) and 19,20-DHDP are Significantly Elevated in Non-Proliferative and Proliferative Diabetic Retinopathy

Consistent with observations in the postnatal murine retina,⁶ the sEH was expressed throughout the different layers of the healthy retina and largely colocalized with glutamine synthetase, indicating expression in Müller glia cells (FIG. 1a). Diabetic retinopathy was studied in male Ins2^{Akita} mice which carry a single amino acid substitution in the insulin 2 gene that causes misfolding of the protein. In line with previous publications,⁸ male Ins2^{Akita} mice developed significant hyperglycemia as early as 4 weeks of age and in retinas from 12 month old Ins2^{Akita} mice with diabetic retinopathy sEH expression and activity were significantly increased (FIG. 1a-c). The increase in retinal sEH expression pre-dated the development of retinopathy and was detectable in 3 month old Ins2^{Akita} mice (FIG. 2) and increased in retinas from wild-type animals fed a high fat diet for 20 weeks that displayed hyperglycemia but no signs of retinopathy (FIG. 1d-e). Levels of 19,20-DHDP were significantly elevated in eyes from Ins2^{Akita} mice and were attenuated by treating animals with the specific sEH inhibitor; trans-4-[4-(3-adamantan-1-ylureido)cyclohexyloxy]-benzoic acid (t-AUCB in drinking water)⁹, from the age of 6 weeks (FIG. 10. Other sEH substrates and products were not significantly affected by diabetes (FIG. 3) and the sEH inhibitor did not affect body weight, fasting blood glucose, blood pressure or heart rate in the animals studied (FIG. 4). sEH expression was also increased in retinas from patients with non-proliferative diabetic retinopathy (FIG. 1g, see Table 1a for patient characteristics), and increased with disease severity (FIG. 5). It was only possible to obtain samples of vitreous humor from patients with proliferative diabetic retinopathy and at this later stage of the disease levels of the sEH substrate; epoxydocosapentaenoic acid (19,20-EDP), and the sEH product; 19,20-DHDP, were significantly higher in samples from patients with diabetic retinopathy than those with idiopathic macular hole but without diabetes and retinopathy (FIG. 1h; see Table 1b for patient characteristics).

TABLE 1a
Characteristics of retina donors;

| | Sample | Gender | | | DM Type | |
|---|---|---|---|---|---|---|
| Pathology | size | Male | Female | Age | Type 1 | Type 2 |
| non-DR | 6 | 4 | 2 | 75.7 ± 3.1 | | |
| NPDR-mild | 7 | 4 | 3 | 57.2 ± 13.0 | 2 | 5 |
| NPDR-severe | 6 | 4 | 2 | 62.2 ± 11.8 | 2 | 4 |

DR = diabetic retinopathy,
NPDR = non-proliferative diabetic retinopathy,
DM = diabetes mellitus.

TABLE 1b
Characteristics of vitreous humor donors with
type 2 diabetes mellitus (DM2);

| | sample | Gender | | | | | DM |
|---|---|---|---|---|---|---|---|
| Pathology | size | Male | Female | Age | MEM | IMH | Type 2 |
| non-DR | 14 | 6 | 8 | 58.9 ± 8.0 | 7 | 7 | |
| DR | 17 | 8 | 9 | 57.0 ± 9.9 | | | 17 |

MEM = macular epiretinal membrane;
IMH: Idiopathic macular hole.

### Example 2: Treatment with an sEH Inhibitor Attenuates Non-Proliferative Diabetic Retinopathy

Diabetic retinopathy is associated with a number of characteristic changes that include pericyte loss and enhanced pericyte migration as well as the appearance of acellular capillaries. All of these phenomena were observed in retinas from Ins2^{Akita} mice, without changes in the numbers of endothelial cells in areas unaffected by vasoregression (FIG. 6a-f). Breakdown of the blood-retinal barrier was also evident in the form of vascular leakage, visualized by intravenous injection with FITC-labelled BSA (FIG. 6g). Chronic treatment with the sEH inhibitor, however, clearly attenuated the hallmarks of diabetic retinopathy and maintained blood-retinal barrier integrity.

### Example 3: Intercellular Junctions are Weakened by 19,20-DHDP

Vascular permeability is largely determined by the integrity of endothelial cell tight junctions and particularly by the surface expression of VE-cadherin¹⁰. VE-cadherin staining clearly demarcated lateral membranes of endothelial cells in vessels from the superficial and deep layers in retinas from wild-type mice (FIG. 7a). However, the continuity of the signal was disrupted in retinas from Ins2^{Akita} mice in which distinct areas demonstrated only a weak punctate staining. In diabetic mice the apparent dissolution of inter-endothelial adherens junctions was evident in the larger vessels of the primary vascular layer as well as in the deeper capillary layer and was normalized by sEH inhibitor treatment.

The disruption of VE-cadherin at the endothelial cell membrane is a characteristic of diabetic retinopathy¹¹, therefore, the effects of 19,20-EDP and 19,20-DHDP on VE-cadherin were assessed in cultured endothelial cells. As in the in vivo situation, VE-cadherin staining clearly labelled the boundaries of confluent endothelial cells, and in the presence of 19,20-DHDP but not its precursor, VE-cadherin staining was discontinuous (FIG. 7b). Functionally, 19,20-DHDP increased endothelial cell permeability as determined by permeability to dextran in confluent cultures of human endothelial cells (FIG. 7c) as well as by decreased transendothelial electrical resistance in cultured of mouse brain microvascular endothelial cells (FIG. 7d). The alterations induced by 19,20-DHDP were however less pronounced than those elicited by vascular endothelial cell growth factor (VEGF) which is reported to induce the internalization of VE-cadherin¹². Internalization was also detected in cells incubated with 19,20-DHDP but not 19,20-EDP as assessed using a pre-labelled anti-VE-cadherin antibody that only recognized the extracellular domain of the protein (FIG. 7e) and by immunoprecipitation of internalized as well as surface VE-cadherin (FIG. 7f). Previous studies have shown that a proportion of VE-cadherin is concentrated in lipid rafts fractions of the membrane where it is stabilized by p120 catenin¹³. 19,20-DHDP decreased the recovery of VE-cadherin from endothelial cell lipid rafts while 19,20-EDP was without effect (FIG. 7g).

### Example 4: 19,20-DHDP Induces Pericyte Loss and Motility by Disruptin N-Cadherin Lipid Raft Localization

Pericyte loss is considered a hallmark of early diabetic retinopathy¹⁴, and it is speculated that pericytes are the primarily affected vascular cells, leading to secondary changes of the endothelium^{15,16}. A loss of retinal vascular mural cells was apparent in retinas from Ins2^{Akita} mice as the loss of smooth muscle actin (see FIG. 6a) and desmin coverage (FIG. 8a). Interestingly, the loss of desmin positive circumferential cells also coincided with the altered patterning of the underlying N-cadherin that is generally enriched at endothelial cell-pericyte junctions^{15,17}. sEH inhibition prevented the loss of desmin positive cells in retinas from Ins2^{Akita} mice and largely maintained N-cadherin expression. N-Cadherin expression was low in endothelial cell monolayers but clearly detectable in co-cultures of endothelial cells and pericytes and enriched at contact points between the 2 cell types (not shown). In cocultured cells 19,20-DHDP decreased N-cadherin expression while 19,20-EDP and VEGF were without effect (FIG. 8c). The 19,20-DHDP-induced reduction in endothelial cell-pericyte contacts was also associated with an increase in pericyte motility (FIG. 8d), an effect mimicked by the siRNA-mediated downregulation of N-cadherin. Moreover, in an ex vivo retina whole mount model, 19,20-DHDP induced capillary pericyte loss and pericyte migration over a 4 day period, evidenced by the migration of desmin-positive cells away from retinal vessels (FIG. 8e-g). 19,20-EDP was largely ineffective. One possible explanation for the increase in pericyte migration could be related to altered growth factor levels in the diabetic retina, particularly platelet-derived growth factor (PDGF) B and its receptor PDGFR-β which have been linked with pericyte apoptosis in diabetic retinopathyl⁸. Indeed, a reduction in PDGF expression leads to a pericyte deficiency that is independent of diabetes^{16,19}. In the Ins2^{Akita} mice, retinal levels of PDGFB were decreased (not shown); a finding that contrasts with observations made in retinas from rats made diabetic with streptozotocin²⁰. Expression of the PDGFRβ tended to increase in ins2^{Akita} retinas but was not significant. sEH inhibition increased PDGFB expression in retinas from wild-type and Ins2^{Akita} mice but failed to significantly alter PDFG receptor β levels. The ins2^{Akita} model reproduces important features of initiation and early progression of diabetic retinopathy (non-proliferative retinopathy) but does not progress to the proliferative form of the disease that is characterized by elevated growth factor expression and angiogenesis⁴. In keeping with this, no reproducible alterations in VEGF, angiopoietins or the levels of their receptors, or in Notch signaling were detected between diabetic and wild-type littermates. Consistent with our previous report, however, sEH inhibition did tend to increase Hey1 and Jagged 1 expression in both wild-type and Ins2^{Akita} mice⁶.

Mechanistically, docosahexenoic acid and DHDP are thought to exert their effects independently of a receptor by means of insertion into the lipid bilayer, a phenomenon previously linked with the redistribution of membrane cholesterol and proteins from lipid raft fractions to non-lipid raft fractions of the plasma membrane^{21,22}. Indeed, 19,20-DHDP was found to inhibit the γ-secretase and interfere with Notch signaling in the retina by eliciting the redistribution of PS1 out of lipid rafts⁶. Given that PS1 interacts with N-cadherin²³⁻²⁵ as well as VE-cadherin²⁶, an alternative explanation for the pericyte drop-off in diabetes was that the 19,20-DHDP could disrupt the membrane localization of N-cadherin at the endothelial cell-pericyte junction in much the same way it targeted VE-cadherin in endothelial cells to promote alterations in cell-cell adhesion. Indeed, 19,20-DHDP, but not its precursor 19,20-EDP, was able to disrupt the lipid raft association and membrane presentation of N-cadherin in endothelial cell-pericyte cocultures (FIG. 8h), and induce N-cadherin internalization (not shown). Indeed, while PS1 co-precipitated with N-cadherin and VE-cadherin from co-cultures of endothelial cells and pericytes, treatment with 19,20-DHDP attenuated this interaction (FIG. 8i-j). Again the precursor 19,20-EDP was without effect. p120 Catenin was proposed to act as a molecular bridge between γ-secretase and cadherin-catenin complexes²⁷, however, 19,20-DHDP did not influence the association of p120 catenin with either N- or VE-cadherin. VEGF also attenuated the association of PS1 with the cadherins, but its effects were less pronounced that those of 19,20-DHDP and associated with decreased recovery of p120 catenin, indicating a distinct mechanism of action. Finally, the co-localization of PS1 with VE-cadherin and N-cadherin could be visualized in retinas from wild-type mice (FIG. 8k), but was disrupted in the areas from 12 month old Ins2^{Akita} retinas that displayed the hallmarks of retinopathy. Again, sEH inhibitor treatment maintained the co-localization of VE-cadherin and N-cadherin with PS1.

### Example 5: Intravitreal Injection of sEH Antagonists Inhibits sEH Enzymatic Function

To test whether sEH could be inhibited therapeutically in the eye, the sEH-inhibitor 1-(1-propanoylpiperidin-4-yl)-3-[4-(trifluoromethoxy)phenyl]urea (TPPU) was injected into the vitreous of ten week old C57 BL6 mice. The retina of the treated animals was then analyzed for sEH enzymatic products. As can be seen in FIG. 9, intravitreal injection of TPPU significantly reduced 19,20 DHDP concentration in the retina (FIG. 9A) as well as other products of sEH (FIG. 9 A, B).

### Example 6: Overactivity of sEH in the Retina has an Effect in the Vasculature Independent of Diabetes

To determine whether the sEH can be linked to the pathogenesis of diabetic retinopathy independently of diabetes per se the enzyme was overexpressed in the Müller cells in vivo with the aid of an adenoviral approach (FIG. 10A). The overexpression of the sEH in the mouse retina markedly increased the number of migrating pericytes and induced pericyte drop-off and the formation of acellular capillaries (FIG. 10B-D). In the same experiments the overexpression of the epoxide hydrolase dead sEH mutant (mutation of Tyr383 and Tyr466 to phenylalanine or sEH^{ΔEH}) was without effect. The experiments cleary demonstrate that in absence of any predisposition to diabetes, the overactivity of the sEH in the retina can reproduce the early vascular defects that are characteristic of non-proliferative diabetic retinopathy.

### Example 7: Local Application of sEH Inhibitors Attenuate sEH Activity In Vivo

The previous studies demonstrating the effectiveness of sEH inhibitors in preventing the development of non-proliferative diabetic retinopathy were performed using a water-soluble inhibitor that was applied by the drinking water. Given that sEH inhibitors may have unwanted systemic effects e.g. on blood pressure a more local form of application may be beneficial, which is particularly appropriate for diseases of the eye. To determine whether or not the local application of sEH inhibitors could affect the activity of the sEH in the retina a short experiment was performed in which mice were treated with eye drops containing either trans-4-(4-(3adamantan-l-yl-ureido)-cyclohexyloxy)-benzoic acid (t-AUCB), or the second-generation sEH inhibitor 1-trifluoromethoxyphenyl-3-(1-propionylpiperidin-4-yl) urea, N-[1-(1-Oxopropyl)-4piperidinyl]-N′-[4-(trifluoromethoxy)phenyl]-urea (TPPU). The concentration of the inhibitors appearing in either the retina, cornea choroid was determined by LCMS/MS (FIG. 11A-B). Using this approach we could demonstrate that the local application of one of the sEH inhibitors tested i.e. TPPU via eye drops could attenuate the activity of the sEH in the retina (FIG. 11C).

### REFERENCES

1. Yau, J. W. Y. et al. Global prevalence and major risk factors of diabetic retinopathy. Diabetes Care 35, 556-564 (2012).
2. Bourne, R. R. A. et al. Causes of vision loss worldwide, 1990-2010: a systematic analysis. Lancet Glob. Health 1, e339-e349 (2013).
3. Das, A., McGuire, P. G., & Rangasamy, S. Diabetic macular edema: pathophysiology and novel therapeutic targets. Ophthalmology 122, 1375-1394 (2015).
4. Robinson, R. et al. Update on animal models of diabetic retinopathy: from molecular approaches to mice and higher mammals. Dis Model Mech 5, 444-456 (2012).
5. Klaassen, I., Van Noorden, C.J.F., & Schlingemann, R. O. Molecular basis of the inner blood-retinal barrier and its breakdown in diabetic macular edema and other pathological conditions. Prog. Retin. Eye Res. 34, 19-48 (2013).
6. Hu, J. et al. Müller glia cells regulate Notch signaling and retinal angiogenesis via the generation of 19,20-dihydroxydocosapentaenoic acid. J. Exp. Med. 211, 281-295 (2014).
7. Dewey, S. et al. Proteomic analysis of hearts from Akita mice suggests that increases in soluble epoxide hydrolase and antioxidative programming are key changes in early stages of diabetic cardiomyopathy. J. Proteome Res. 12, 3920-3933 (2013).
8. Barber, A. J. et al. The Ins2(Akita) mouse as a model of early retinal complications in diabetes. Invest. Ophthalmol. Vis. Sci. 46, 2210-2218 (2005).
9. Hwang, S. H. et al. Orally bioavailable potent soluble epoxide hydrolase inhibitors. J Med. Chem 50, 3825-3840 (2007).
10. Giannotta, M., Trani, M., & Dejana, E. VE-cadherin and endothelial adherens junctions: active guardians of vascular integrity. Developmental Cell 26, 441-454 (2013).
11. Navaratna, D., McGuire, P. G., Menicucci, G., & Das, A. Proteolytic degradation of VE-cadherin alters the blood-retinal barrier in diabetes. Diabetes 56, 2380-2387 (2007).
12. Gavard, J. & Gutkind, J. S. VEGF controls endothelial-cell permeability by promoting the b-arrestin-dependent endocytosis of VE-cadherin. Nat Cell Biol 8, 1223-1234 (2006).
13. Gentil-dit-Maurin, A. et al. Unraveling the distinct distributions of VE- and N-cadherins in endothelial cells: a key role for p120-catenin. Exp. Cell Res. 316, 2587-2599 (2010).
14. Yafai, J. et al. Retinal endothelial angiogenic activity: effects of hypoxia and glial (Müller) cells. Microcirculation 11, 577-586 (2004).
15. Gerhardt, H. & Betsholtz, C. Endothelial-pericyte interactions in angiogenesis. Cell Tissue Res 314, 15-23 (2003).
16. Hammes, H. P. et al. Pericytes and the pathogenesis of diabetic retinopathy. Diabetes 51, 3107-3112 (2002).
17. Gerhardt, H., Wolburg, H., & Redies, C. N-cadherin mediates pericytic-endothelial interaction during brain angiogenesis in the chicken. Dev. Dyn. 218, 472-479 (2000).
18. Geraldes, P. et al. Activation of PKC-d and SHP-1 by hyperglycemia causes vascular cell apoptosis and diabetic retinopathy. Nat Med 15, 1298-1306 (2009).
19. Enge, M. et al. Endothelium-specific platelet-derived growth factor-B ablation mimics diabetic retinopathy. The EMBO Journal 21, 4307-4316 (2002).
20. Yokota, T. et al. Role of protein kinase C on the expression of platelet-derived growth factor and endothelin-1 in the retina of diabetic rats and cultured retinal capillary pericytes. Diabetes 52, 838-845 (2003).
21. Wassall, S. R. & Stillwell, W. Polyunsaturated fatty acid-cholesterol interactions: domain formation in membranes. Biochim. Biophys. Acta 1788, 24-32 (2009).
22. Wassall, S. R. & Stillwell, W. Docosahexaenoic acid domains: the ultimate non-raft membrane domain. Chem. Phys. Lipids 153, 57-63 (2008).
23. Georgakopoulos, A. et al. Presenilin-1 forms complexes with the cadherin/catenin cell-cell adhesion system and is recruited to intercellular and synaptic contacts. Mol Cell 4, 893-902 (1999).
24. Baki, L. et al. Presenilin-1 binds cytoplasmic epithelial cadherin, inhibits cadherin/p120 association, and regulates stability and function of the cadherin/catenin adhesion complex. Proc. Natl. Acad. Sci. U. S. A 98, 2381-2386 (2001).
25. Serban, G. et al. Cadherins mediate both the association between PS1 and b-catenin and the effects of PS1 on b-catenin stability. J Biol Chem. 280, 36007-36012 (2005).
26. Cai, J. et al. PEDF regulates vascular permeability by a g-secretase-mediated pathway. PLoS ONE 6, e21164 (2011).
27. Kouchi, Z. et al. p120 catenin recruits cadherins to g-secretase and inhibits production of Ab peptide. J Biol Chem. 284, 1954-1961 (2009).
28. Morisseau, C. et al. Structural refinement of inhibitors of urea-based soluble epoxide hydrolases. Biochem. Pharmacol. 63, 1599-1608 (2002).
29. Calabria, A. R. et al. Puromycin-purified rat brain microvascular endothelial cell cultures exhibit improved barrier properties in response to glucocorticoid induction. J Neurochem. 97, 922-933 (2006).
30. Czupalla, C. J., Liebner, S., & Devraj, K. In vitro models of the blood-brain barrier. Methods Mol Biol 1135, 415-437 (2014).
31. Bess, E., Fisslthaler, B., Frömel, T., & Fleming, I. Nitric oxide-induced activation of the AMP-activated protein kinase a2 subunit attenuates IkB kinase activity and inflammatory responses in endothelial cells. PLoS ONE 6, e20848 (2011).
32. Kovacevic, I. et al. The F-BAR protein NOSTRIN participates in FGF signal transduction and vascular development. EMBO J 31, 3309-3322 (2012).
33. Hammes, H. P. et al. Aminoguanidine treatment inhibits the development of experimental diabetic retinopathy. Proc. Natl. Acad. Sci. U. S. A 88, 11555-11558 (1991).
34. Pokreisz, P. et al. Cytochrome P450 epoxygenase gene function in hypoxic pulmonary vasoconstriction and pulmonary vascular remodeling. Hypertension 47, 762-770 (2006).
35. Barbosa-Sicard, E. et al. Inhibition of the soluble epoxide hydrolase by tyrosine nitration. J. Biol. Chem. 284, 28156-28163 (2009).
36. Rivera, J. et al. Measurement of 20-hydroxyeicosatetraenoic acid in human urine by gas chromatography-mass spectrometry. Clin Chem. 50, 224-226 (2004).
37. Busse, R. & Lamontagne, D. Endothelium-derived bradykinin is responsible for the increase in calcium produced by angiotensin-converting enzyme inhibitors in human endothelial cells. Naunyn Schmiedebergs Arch. Pharmacol. 344, 126-129 (1991).
38. Paolinelli, R. et al. Wnt activation of immortalized brain endothelial cells as a tool for generating a standardized model of the blood brain barrier in vitro. PLoS ONE 8, e70233 (2013).
39. Taddei, A. et al. Endothelial adherens junctions control tight junctions by VEcadherin-mediated upregulation of claudin-5. Nat Cell Biol 10, 923-934 (2008).
40. Xiao, K. et al. p120-Catenin regulates clathrin-dependent endocytosis of VE-cadherin. Mol Biol Cell 16, 5141-5151 (2005).
41. Fleming, I., Fisslthaler, B., Dixit, M., & Busse, R. Role of PECAM-1 in the shear-stress-induced activation of Akt and the endothelial nitric oxide synthase (eNOS) in endothelial cells. J. Cell Sci. 118, 4103-4111 (2005).
42. Vetrivel, K. S. et al. Association of g-secretase with lipid rafts in post-Golgi and endosome membranes. J Biol Chem 279, 44945-44954 (2004).

1. A method for treating an eye disease associated with the blood-retinal barrier, wherein said method comprises administering to a subject in need of such treatment, an inhibitor or antagonist of 19,20-dihydroxydocosapentaenoic acid (19,20-DHDP).

2. The method according to claim 1, wherein the inhibitor or antagonist of 19,20-DHDP is a substance that reduces the biochemical synthesis and/or biological actions of 19,20-DHDP, or wherein the inhibitor or antagonist of 19,20-DHDP is an inhibitor or antagonist of soluble epoxide hydrolase (sEH).

3. The method according to claim 1, wherein the eye disease associated with the blood-retinal barrier is characterized by increased pericyte motility and/or pericyte loss in a retina.

4. The method according to claim 1, wherein the eye disease associated with the blood-retinal barrier is diabetic retinopathy.

5. The method according to claim 1, wherein the eye disease associated with the blood-retinal barrier is a non-proliferative diabetic retinopathy.

6. (canceled)

7. The method according to claim 1, wherein the subject further suffers from a metabolic disorder.

8. The method according to claim 1, wherein the subject does not suffer from a proliferative form of diabetic retinopathy.

9. A pharmaceutical composition for use in the treatment of an eye disease associated with the blood-retinal barrier, comprising an inhibitor or antagonist of 19,20-dihydroxydocosapentaenoic acid (19,20-DHDP) and a pharmaceutically acceptable carrier and/or excipient.

10. An in vitro method selected from:
A) an in vitro method for diagnosing a non-proliferative diabetic retinopathy in a subject comprising determining the level of sEH, and/or 19,20-DHDP in a vitreous sample from the subject, wherein an elevated level of sEH and/or 19,20-DHDP in the sample from the subject compared to a control sample or value indicates a non-proliferative diabetic retinopathy; and
B) an in vitro method for monitoring a subject for the development of a diabetic retinopathy, wherein the method comprises determining the level of sEH, and/or 19,20-DHDP in vitreous samples obtained from the subject at at least one earlier and at at least one later time point, wherein an increase of the level sEH, and/or 19,20-DHDP in the vitreous sample obtained at the at least one later time point compared to the vitreous sample obtained at the at least one earlier time point indicates the development of a non-proliferative diabetic retinopathy in the subject.

11. (canceled)

12. The in vitro method, according to claim 10, wherein said method is an in vitro method for diagnosing a non-proliferative diabetic retinopathy in a subject comprising determining the level of sEH, and/or 19,20-DHDP in a vitreous sample from the subject, wherein an elevated level of sEH and/or 19,20-DHDP in the sample from the subject compared to a control sample or value indicates a non-proliferative diabetic retinopathy.

13. The in vitro method, according to claim 10, wherein said method is an in vitro method for monitoring a subject for the development of a diabetic retinopathy, wherein the method comprises determining the level of sEH, and/or 19,20-DHDP in vitreous samples obtained from the subject at at least one earlier and at at least one later time point, wherein an increase of the level sEH, and/or 19,20-DHDP in the vitreous sample obtained at the at least one later time point compared to the vitreous sample obtained at the at least one earlier time point indicates the development of a non-proliferative diabetic retinopathy in the subject.

14. The method, according to claim 1, wherein the eye disease is an early stage diabetic retinopathy.

15. The method, according to claim 7, wherein the metabolic disorder is type I or type II diabetes.
